(19) United States
(12) Patent Application Publication
(10) Pub. No.: US 2004/0265252 A1
(43) Pub. Date: Dec. 30, 2004

(54) Compounds stimulating and inhibiting melanin formation, and methods for screening these compounds

(75) Inventors: Seth Orlow, New York, NY (US); Young-Tae Chang, New York, NY (US)

Correspondence Address: BROWDY AND NEIMARK, P.L.L.C., 624 NINTH STREET, NW, SUITE 300, WASHINGTON, DC, 20001-5303, US

(73) Assignee: New York University, New York, NY

(21) Appl. No.: 10/821,981

(22) Filed: Apr. 12, 2004

Related U.S. Application Data
(60) Provisional application No. 60/461,804, filed on Apr. 11, 2003.

(51) Int. Cl. C12Q 1/00; A61K 7/135

(52) U.S. Cl. 424/62; 435/4

(57) ABSTRACT

Trisubstituted triazine compounds are screened for pigment stimulating ability or pigment inhibiting ability. Compounds which bind ATPase increase melanin in the skin or hair. Compounds found to be active are used for screening for other active compounds. Active compounds are used for increasing melanin in the skin or hair, or for decreasing melanin in the skin or hair.

### CROSS-REFERENCE TO RELATED APPLICATIONS

This application claims priority from provisional application Ser. No. 60/461,804, filed 11 Apr. 2003, the entire contents of which are hereby incorporated by reference.

### FIELD OF THE INVENTION

The present invention relates to compounds that can be used for inhibiting or for stimulating the production of melanin, as well as for methods for screening compounds and libraries of compounds to determine their efficacy for inhibiting or stimulating melanin production.

### BACKGROUND OF THE INVENTION

Melanin is a dark pigment that protects against ultraviolet radiation and provides decoration in the skin, eyes, hair, and fur of animals. Defects in the production of melanin result in pigmentation deficiencies such as albinism.

Increasing skin pigmentation would be desirable both to increase melanin protection from ultraviolet radiation without exposing the skin to UV light, to correct hypopigmentation disorders, and for cosmetic purposes to achieve a “safe” tan or to darken hair color. Decreasing pigmentation would be desirable to treat disorders such as melasma, chloasma, post-inflammatory hyperpigmentation, solar lentigines, and the like.

Melanocytes synthesize melanin inside of specialized organelles called melanosomes (reviewed in Orlow, S. J., 1998, in The Pigmentary System: Physiology and Pathophysiology 97, Oxford University Press, New York, Nordlund et al., eds). Melanosomes are formed by the fusion of two types of vesicles.

Defects in the production of melanin result in pigmentation deficiencies such as albinism. Genetic analysis of abnormally pigmented strains of mice has identified more than 60 genes necessary for the normal production of melanin (reviewed in Silvers, W. K., 1979, The Coat Colors of Mice: A Model for Mammalian Gene Action and Interaction, Springer-Berlag, Basel). One of these genes encodes the enzyme tyrosinase. Tyrosinase protein is a multi-functional enzyme that catalyzes several steps in the production of melanin; tyrosinase activities include the rate-limiting steps of converting tyrosine to dihydroxyphenylalanine (DOPA), and DOPA to dopaquinone (reviewed in Lerner, A. B. et al., 1950, Physiolo. Rev. 30:90-126), as well as the oxidation of 5,6-dihydroxyindole to 5,6-indolequinone (Korner and Pawelek, 1982, Science 217:1163-1165).

Both humans and mice lacking tyrosinase activity suffer a severe form of albinism. Two tyrosinase-related proteins, TRP-1, encoded by the mouse brown gene, and TRP-2. encoded by the mouse slaty gene, also are important for melanogenesis (reviewed in Hearing, V. J., 1993, Am. J. Hum. Genet. 52:1-7). Each of the TRP proteins shares about 40% sequence identity with tyrosinase and with each other. Each of these three enzymes (tyrosinase, TRP-1 and TRP-2) is predicted to contain one transmembrane domain.

Another protein that is important for melanin production is the P protein. In mice, it is the product of the pink-eye dilution (p) gene. In humans, it is the product of the P gene. Humans lacking P protein function suffer from type II oculocutaneous albinism (Durham-Pierre, D. et al., 1994 Nature Genetics 7: 176-179). Mice lacking the p gene produce significantly less melanin than wild-type mice. A wild-type human P gene, but not a mutant human P gene, can complement the hypopigmented phenotype of p-null mouse melanocytes (Sviderskaya, E. V., et al., 1997, J. Invest. Dermatol. 190:30-34). P protein is apparently needed for production of brown-black eumelanin, but not for yellow-red pheomelanin (Lamoreux, M. L., et al., 1995, Pigment Cell Res. 8: 263-70).

The P protein is predicted to contain 12 membrane spanning domains (Gardner, J. M., et al., 1992, Science 257: 112-124).

Several authors have suggested that P protein acts as a tyrosine transporter by pumping tyrosine into the melanosome where it is converted into melanin by tyrosinase activity (see, e.g., Rinchik, E. M. et al., 1993, Nature 361: 72-76). First, the P protein bears some resemblance to transport proteins found in prokaryotes. Second, cultured p-null mutant mouse melanocytes, which produce much less melanin than cultured wild-type mouse melanocytes, make increased levels of melanin when high concentrations of tyrosine are added to the cells' growth medium (Sviderskaya, E. V. et al., above; Rosemblat, S., et al., 1998, Exp. Cell Res. 239: 3440352). However, contradicting this suggestion, it has been found that tyrosine uptake by melanosomes is virtually the same in p-null and wild-type melanocytes (Gahl, W. A., et al., 1995, Pigment Cell Res. 8:229-233). This observation has led other authors to hypothesize that P protein is necessary for the transport into melanosomes of some other small molecule necessary for melanogenesis (summarized in Brilliant, M. H. et al., 1998, above).

Other authors have speculated that P protein plays a structural role in melanosomes (Lamoreux, M. L. et al., above). The integrity of melanosomes is compromised in cells lacking P protein. Tyrosinase activity, and therefore melanin production, is greatly decreased in these defective melanosomes.

Specifically, tyrosinase activity levels in melanocyte extracts of skin and eyes from p-null mice are lower than such extracts from wild-type mice (Lamoreux, M. L., et al., above; Chiu, E., et al., 1993, Exp. Eye Res. 57: 301-305). Moreover, levels of tyrosinase, TRP-1 and TRP-2 proteins are lower in p-null tissue extracts than in wild-type extracts (Rosemblat, S. et al., 1998, above). Additionally, a much greater percentage of tyrosinase, TRP-1 and TRP-2 proteins are found in their monomeric forms rather than as part of a high molecular weight complex, in p-null tissue extracts than in wild-type extracts (Lamoreux, M. L., above; Chiu, E., et al., above)., Tyrosinase, TRP-1 and TRP-2 are all rapidly degraded in the ocular tissue of p-null mice (Chiu, E., et al., above). Finally, several authors have observed that melanosomes in p-null tissues and cultured melanocytes are abnormal (Russell, E. S., 1949, Genetics 34: 146-166); Rosemblat, S. et al., 1998, above). In p-mutant melanocytes from mouse eye, very few melanosomes are observed (Orlow, S. J., et al., 1999, Exp. Eye Res. 68: 147-154). In cultured mutant melanocytes, a greater than normal number of melanosomes is present, but they are smaller than those seen in wild-type melanocytes (Rosemblat, S., et al., 1998). Thus, although P protein is known to be critical for the production of normal amounts of melanin in the skin, hair, and eyes, the function of the P protein in this process has remained elusive. Instead, researchers have looked to other molecular targets for inhibition studies. For example, tyrosinase's well-characterized enzymatic activity, amenability to biochemical analysis, and pivotal role in melanogenesis have made it an inviting target for inhibition studies (see, e.g., Tasaka, K. et al., 1998, Meth. Find. Expl. Clin. Pharmacol 20:99-109; Iida, K. et al., 1995, Planta Med. 61:425-428; Reish, O., et al., 1995, Am. J. Hum. Genet. 57:127-132).

For many individuals of all ages, the inappropriate production of overproduction of melanin is a cosmetic problem. By way of example, many children develop freckles after exposure to the sun. For individuals in middle or advanced age, chloasma, freckles, and pigmentary deposits after sunburn tend to occur or increase in frequency. In addition, these pigment deposits do not disappear quickly and are more likely to become permanent with advancing age.

A number of products have been developed to effect a decrease in skin pigmentation. One such product contains hydroquinone, a well known active substance for skin de-pigmentation, as described in U.S. Patent No. 6,139,854. However, hydroquinone can have serious side effects if applied over a long period of time. For example the application of hydroquinone to skin may lead to permanent de-pigmentation, and thus to increased photosensitivity of the skin when exposed to ultraviolet light. For that reason, in some countries, hydroquinone is only allowed to be used for skin de-pigmentation in limited concentrations. In other countries, the product is banned completely for this application. Hydroquinone is a very poor inhibitor of tyrosinase, the rate limiting enzyme in pigmentation. Hydroquinone can be administered in combination with cortisone (which can thin the skin and cause other problems from facial administration), retinoic acid (an irritant), or glycolic acid (an irritant) to increase the efficacy of hydroquinone.

A variety of other substances have been proposed for control or inhibition of skin pigmentation. Almost all of these substances work by either bleaching existing pigment or preventing new pigment synthesis by inhibiting the activity of tyrosinase, the principle rate limiting enzyme in the production of melanin. For example U.S. Pat. No. 6,123,959 described the use of aqueous compositions comprising liposomes and at least one competitive inhibitor of an enzyme for the synthesis of melanin. U.S. Pat. No. 5,132,740 describes the use of certain resorcinol derivatives a skin lightening agents. WO 99/64025 describes compositions for skin lightening which contain tyrosinase inhibiting extracts from dicotyledonous plant species indigenous to Canada. U.S. Pat. No. 5,580,549, described an external preparation for skin lightening comprising 2-hydroxybenzoic acid derivatives and salts thereof as inhibitors of tyrosinase. WO 99/09011 describes an agent for inhibiting skin erythema and/or skin pigmentation, containing at least one carbostyril derivative and salts thereof. U.S. Pat. Nos. 5,214,028 and 5,389,611, describe lactoferrin hydrolyzates for use as tyrosinase inhibitory agents.

Manga, in WO 02 98347, describes methods for identifying compounds that inhibit melanogenesis in melanogenic cells, more particularly, compounds that inhibit or enhance P protein function. This method is based, in part, on the discovery that P protein function is required for proper cellular localization of tyrosinase and other melanosomal proteins, and is required for both full tyrosinase activity and melanogenesis in melanogenic cell types.

Orlow et al., in WO 01 1131, describe screens for identifying compounds that inhibit or increase melanogenesis in melanogenic cells based upon the discovery that some compounds that inhibit melanogenesis do so by causing a mislocalization of tyrosinase, the key enzyme in melanin synthesis.

Chemical library screening can be used to find a phenotypic change by targeting specific gene products, that is, proteins; this technique is called chemical genetics. In chemical genetics, one chemical compound may specifically inhibit or activate one target protein (for purposes of illustration, called “protein A”). Thus, the compound is equivalent to the gene knock-out or over-expression of the corresponding gene A, as in conventional genetics.

Combinatorial library techniques facilitate the synthesis of many molecules. These techniques can be combined with high throughput screening (HTS) to screen many compounds to discover a novel, small molecule in the first step of chemical genetics study. Once one finds an intriguing small molecule, here referred to as “molecule A”, that induces a novel phenotype in cells or in an organism, the next step is to identify the target protein and the biochemical pathways involved. An affinity matrix on bead, or a tagged molecule (photoaffinity, chemical affinity, biotin or fluorescence) obtained by modifying molecule A, is commonly used for identifying the target protein. The target can be fished out by binding affinity of the proteins to the immobilized molecule, followed by separation on gel and sequencing by tandem mass spectrometry (MS-MS) techniques. As the affinity matrix isolation usually gives multiple proteins, including non-specific binders, it is best to compare the gel results with those of control matrices side by side. Desirable control matrices will be obtained from structurally similar molecules to molecule A which are inactive. The proteins that bind only to the active affinity matrix, without binding to the control matrices, are promising target candidates. The candidate proteins are then purified and screened in vitro with molecule A to confirm that the isolated protein is truly protein A.

As a whole, successful chemical genetics work will identify a novel gene product (i.e., protein A), and its on or off switch, small molecule pairs. By analyzing the phenotype change, the function of protein A, which is the expression product of gene A, will be discerned. At the same time, the identified small molecule key, molecule A, is a useful biochemical tool to regulate the pathway of protein A, and may be a promising drug candidate as well.

Unfortunately, the current approach of chemical genetics intrinsically contains a very difficult step, that of modifying molecule A into an affinity molecule. In order to add a linker to molecule A without adversely affecting its activity, a thorough structure-activity relationship (SAR) study of molecule A is required to find a proper site for linker addition. This site is probably a site of molecule A exposed to the solvent direction from a binding pocket in protein A. This procedure is, in many cases, extremely cumbersome, and sometimes is even completely impossible.

### SUMMARY OF THE INVENTION

It is an object of the present invention to overcome the aforesaid deficiencies in the prior art.

It is another object of the present invention to provide methods for screening compounds that can stimulate or inhibit pigmentation in melanocytes.

It is a further object of the present invention to provide libraries of compounds for stimulating or inhibiting pigmentation in melanocytes.

It is a further object of the present invention to provide compounds for treating hypopigmentation or hyperpigmentation.

It is yet another object of the present invention to provide compounds and methods for protecting skin from ultraviolet rays.

It is still another object of the present invention to provide methods for synthesizing compounds which inhibit or stimulate pigmentation in melanocytes.

It is yet another object of the present invention to identify proteins that interact with triazine compounds, and then to use these proteins to find other compounds that interact with the proteins.

It is a further object of the present invention to identify compounds that interact with mitochondrial ATPase to modulate pigmentation.

It is another object of the present invention to identify compounds that affect levels of mRNA for one or more mitochondrial ATPase units.

It is yet another object of the present invention to identify compounds that interact with prohibitin or which alter levels of prohibitin mRNA to modulate pigmentation.

According to the present invention, chemical genetics is used to investigate cellular pathways and to identify novel gene products by conditionally modulating protein function using small molecules based on trisubstituted triazines. The present invention provides large and structurally diverse chemical libraries of these compounds that are subsequently screened for a desired cellular phenotype.

For purposes of the present invention, “inhibiting” or “stimulating” will also be referred to as “altering.”

One group of compounds of the present invention includes pigment enhancing or inhibiting compounds obtained from a triazine based chemical library. Active compounds were structurally derivatized to maximize their cellular effectiveness. This new class of compounds affects melanin production, tyrosinase, activity, and tyrosinase, TRP-1 and TRP-2 protein levels in a dose-dependent manner at nontoxic effective concentrations. Treating melanocytes with these chemicals in combination with the known promoter isobutylmethylxanthine (IBMX) resulted in a further dramatic increase in melanin production, tyrosinase activity, and tyrosinase, TRP-1 and TRP-2 protein levels, as did the combination of these trisubstituted triazines with compounds that increase cyclic AMP levels and/or that stimulate melanotropin receptors or that inhibit MAP kinase.

In addition to the triazine based compounds which have been found to enhance or inhibit pigmentation, it has now been discovered that compounds that interact with mitochondrial ATPase also promote pigmentation. Among these compounds, in addition to the triazine compounds are mitochondrial ATPase inhibitors such as oligomycins and aurovertins. The aurovertins inhibit proton-pumping F₁F₀ATPase by binding to beta-subunits in its F₁ catalytic sector. Aurovertins B and D have identical biological properties and are more potent than aurovertin A. The oligomycins also inhibit proton-pumping by F₁F₀ ATPase. Therefore, all compounds that inhibit proton pumping of F₁F₀ ATPase can be used to affect pigmentation. However, one skilled in the art, without undue experimentation, can readily determine other compounds that interact or bind with mitochondrial ATPase using conventional techniques.

The trisubstituted triazines of the present invention can be used with cAMP elevating agents and MAP kinase inhibitors, which act synergistically with the triazines to increase pigmentation. Additionally, protein kinase C inhibitors augment triazine-induced pigmentation.

Protein kinase A inhibitors block the action of compounds which increase melanin production such as V28, IBMX, etc. Thus, protein kinase A inhibitors can be used in combination with inhibitors identified from the trisubstituted triazine library of the present invention to augment their ability to inhibit pigmentation, as well as with other inhibitors of mitochondrial ATPase or with drugs that block prohibitin formation.

### BRIEF DESCRIPTION OF THE DRAWINGS

FIGS. 1A-D show that the novel class of triazine based molecules according to the present invention are potent stimulators of melanin production in cultured melan-a melanocytes, and act synergistically with the known promoter IBMX.

FIG. 1A shows TG library structures. Lead compounds TGV28, also called V28 and TGV30, also called V30 were identified from the primary pharmacologic screen.

FIG. 1B is a photograph of cultured melan-a cells showing (a) untreated), (b) cells treated with IBMX. (100 micromolar), (c) V28, 5 Micromolar, (d) V28 5 Micromolar plus IBMX, 100 micromolar.

FIG. 1C is a digital image quantification of FIG. 1B using SCION IMAGE® and ADOBE PHOTOSHOP®.

FIG. 1D shows the results of a melanin assay. The numbers above the bars indicate the percent increase in melanin production relative to the untreated control. For this assay, melan-a melanocytes were cultured in DMEM supplements with 10% FCS, 1% L-glutamine, 1% penicillin/streptomycin, and 40 nM TPA. The cultures were maintained at 37° C., 5% CO₂. The cells were seeded at 5×10⁴ cells per well in 24 well plates and treated for 72 hours. Photographs were taken after 72 hours at 40× magnification. The cells were harvested, centrifuged, and the protein was extracted from the pellet with 1:1 ethanol:ether. The melanin pellets were solubilized in 20% DMSO/2N NaOH. Samples were transferred to a 96-well plate and the optical density was read at 490 nm using a Biorad 550 spectrophotometer.

FIGS. 2A-C illustrate that compound V28 increases cell-associated tyrosinase activity in a dose=dependent manner.

In FIG. 2A, tyrosinase assay on cell lysates derived from whole cells shows a dose-dependent increase in enzyme activity in cells treated with V28.

FIG. 2B shows the results of a tyrosinase assay on conditioned media, showing a large increase in secreted enzyme activity in cells treated with IBMX and only a marginal increase with treatment with V28.

FIG. 2C shows that tyrosinase assay on pre-lysed cells demonstrates a significant decrease in tyrosinase activity with treatment with the tyrosinase inhibitor phenythiourea (PTU) and only a marginal change with treatment with IBMX and V28.

FIG. 3 shows the reaction scheme for synthesizing the library of compounds of the present invention.

FIG. 4 is a photograph of a silver-stained 5% to 15% gradient SDS-PAGE gel illustrating V28-specific protein binding.

FIG. 5 illustrates that V28 binds prohibitin specifically and with high affinity.

FIG. 6 shows that mitochondrial ATPase is the target of PPA.

FIG. 7 shows that mitochondrial ATPase is the target of PPA.

FIG. 8 illustrates that aurovertin and PPA6 do not compete for binding of ATPase to PPA-Affi.

FIG. 9 shows that both the beta subunit and oligomycin sensitivity conferring protein are specifically bound to a PPA-Affi matrix and are eluted by oligomycin.

FIG. 10 is the same as FIG. 9 but with antibody to the d subunit of ATPase.

FIG. 11 shows that PPA, oligomycin, and aurovertin all promote pigmentation.

FIG. 12 shows that PPA and oligomycin promote pigmentation.

FIG. 13 show aurovertin promotes pigmentation.

FIG. 14 shows the structures of PPA, also known as PPJ7, and ADA. It should be noted that PPA6 is inactive.

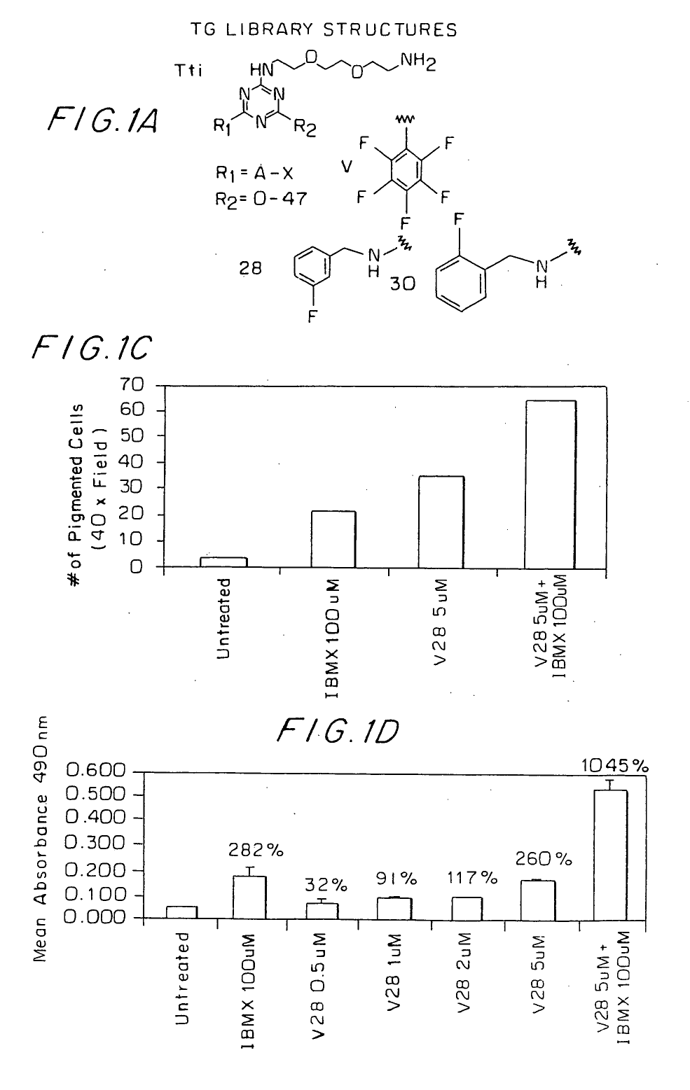

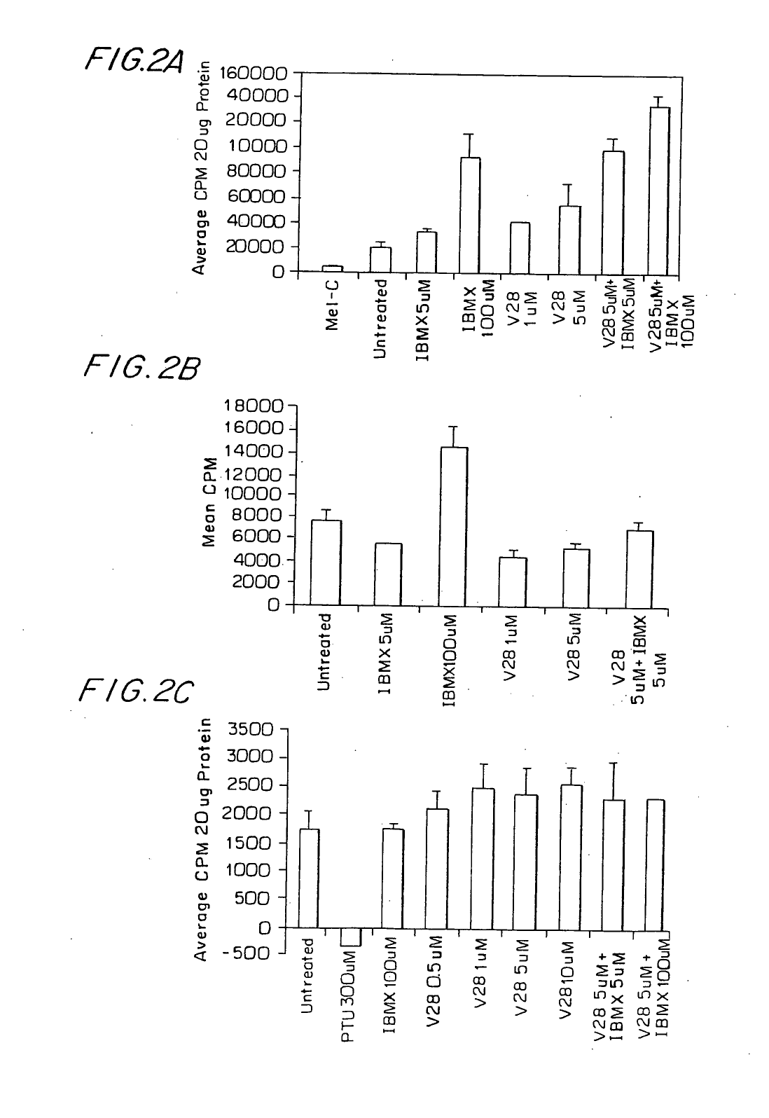

### DETAILED DESCRIPTION OF THE INVENTION

The present invention provides potent novel compounds identified by screening a chemical library based upon a symmetric triazine core for those compounds that can stimulate or inhibit pigmentation in cultured melanocytes. Structure features leading to activity have been defined by combinatorial chemistry.

In addition, compounds which interact with mitochondrial ATPase can also be used to affect pigmentation in melanocytes.

Many of the triazine and other compounds of the present invention have linkers which enable them to be readily immobilized on an affinity matrix or otherwise be combined with a variety of probes for isolating or identifying proteins with which they interact. Those compounds that lack a linker can be reacted with a suitable moiety to provide a linker arm so that the triazine molecule can be immobilized on an affinity matrix, or can be attached to a marker such as a fluorescent marker. Once a compound has been identified as one that stimulates or inhibits pigmentation, the protein that interacts with that compound can be identified. This target protein can then be used to identify other compounds that interact with the target. Thus the compounds of the present invention can be used through their interaction with proteins to identify still other compounds that interact with these proteins.

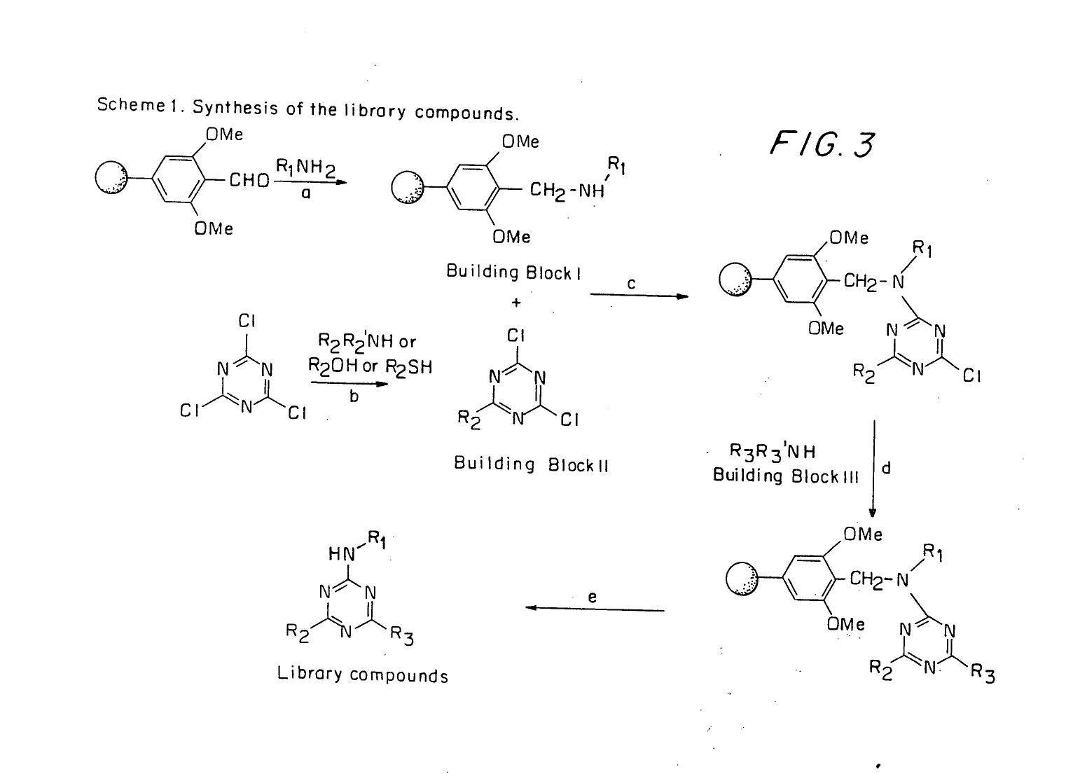

The library compounds are synthesized by first preparing three building blocks separately and then assembling them by orthogonal reaction, as shown in FIG. 3. The reagents and conditions are:

a. RNH₂ (5 equivalents), 2% acetic acid in THF, rt, for one hour, followed by NaB(OAc)3H, 7 equivalents, rt, for twelve hours;

b. R₂NH₂, R₂OH or R₂SH (1 equivalent) in THF, 0° C., for one hour;

c. Building block II (4 equivalents) in 60° C. THF for one hour, diisopropylethylamine (DIEA);

d. R₃R₃NH or R₃SH, DIEA, NMP, n-butanol 1:1 for three hours at 120° C.;

e. 5% TFA in dichloromethane for 10 minutes.

Building Block I is a PAL aldehyde resin bound to a primary amine via reductive amination reaction. Building Block II is synthesized in solution, and is an amine, alcohol, or thiol attached to cyanuric chloride. Building Block III is a series of primary or secondary amines.

Building block I was synthesized by combining 10 mg of 0.01 mmol of PAL aldehyde resin (purchased from Midwest Bio-Tech, sub. 1.10) with 13.6.mg, 0.055 mmol, 5 eq. Of Boc-linker [2-(2-amino-ethoxy-ethoxyethyl)-carbamic-tert-butyl ester) in 2 ml THF containing 40 microliters of acetic acid, at room temperature. The reaction mixture was stirred for one hour at room temperature, followed by addition of 16 mg (0.077 mmol, 7 equivalents) of sodium triacetoxyborohydride. The reaction mixture was stirred for 12 hours, filtered, and the resin was washed three times in DMF, three times in dichloromethane, three times in methanol, and three times in dichloromethane.

Building block II was synthesized by combining 100 mg (0.543 mmol) cyanuric chloride with 0.05 ml DIEA in 5 ml. THF at 0° C. An amine or alcohol (0/652 mmol, 1.2 eq) was then added to the reaction mixture and stirred for 30 minutes at 0° C. The progress of the reaction was monitored by TLC. At the end of the reaction period, the mixture was filtered through a plug and solvent was removed in vacuo. The compounds were further purified by column chromatography.

Building Block 1 (10 mg) and Building Block II (4 equivalents) were combined together with 4 equivalents of DIEA in 1 ml. THF. The reaction mixture was heated to 60° C. for three hours, filtered, and the resin was washed three times with DMF, three times with dichloromethane, three times with methanol, and three times with dichloromethane.

Building Block III, a primary or secondary amine (4 equivalents) was then combined with the resulting resin (10 mg), 4 equivalents DIEA, in 1:1 butanol-1-methyl-2-pyrrolidinone (NMP). The reaction mixture was heated to 120° C. for three hours, filtered, and the resin was washed three times with DMF, three times with dichloromethane, three times with methanol, and three times with dichloromethane. 5% TFA in dichloromethane (0.5 ml) was then added to the resin and stirred for 30 minutes. The purity and identify of the product were monitored by LC-MS.

The building blocks for use in preparing the trisubstituted triazines comprise:

a. a primary amine of the formula RNH₂, wherein R is selected from the group consisting of substituted or unsubstituted alkyl, substituted or unsubstituted alkenyl, substituted or unsubstituted alkynyl, substituted or unsubstituted aryl, substituted or unsubstituted arylalkyl, and substituted or unsubstituted heteroaryl groups;

b. R₂NH, R₂OH, or R₂SH, wherein R₂ is selected from the group consisting of substituted or unsubstituted alkyl, substituted or unsubstituted alkenyl, substituted or unsubstituted alkynyl, substituted or unsubstituted aryl, substituted or unsubstituted arylalkyl, and substituted or unsubstituted heteroaryl groups; and

c. R₃R₃NH, R₃SH, wherein R₃ is selected from the group consisting of substituted or unsubstituted alkyl, substituted or unsubstituted alkenyl, substituted or unsubstituted alkynyl, substituted or unsubstituted aryl, substituted or unsubstituted arylalkyl, and substituted or unsubstituted heteroaryl groups.

As used herein, alkyl, alkenyl and alkynyl carbon chains, if not specified, contain from 1 to 20 carbon atoms, preferably from 1 to 16 carbon atoms, and are straight or branched. Alkenyl carbon chains of from 1 to 20 carbon atoms preferably contain 1 to 8 double bonds; the alkenyl carbon chains of 1 to 16 carbon atoms preferably contain from 1 to 5 double bonds.

Alkynyl carbon chains of from 1 to 20 carbon atoms preferably contain 1 to 8 triple bonds, and the alkynyl carbon chains of 1 to 16 carbon atoms preferably contain 1 to 5 triple bonds. The alkyl, alkenyl, and alkynyl groups may be. optionally substituted, with one or more groups, preferably alkyl group substituents that may be the same or different. As used herein, lower alkyl, lower alkenyl, and lower alkynyl refer to carbon chains having fewer than or equal to about 6 carbon atoms.

As used herein an alkyl group substituent includes halos, haloalkyl, preferably halo lower alkyl, aryl, hydroxy, alkoxy, aryloxy, alkoxy, alkylthio, arylthio, aralkyloxy, aralkylthio, carboxy, alkoxycarbonyl, oxo, and cycloalkyl.

For the present invention, “cyclic” refers to cyclic groups preferably containing from 3 to 19 carbon atoms, preferably 3 to 10 members, more preferably 5 to 7 members. Cyclic groups include hetero atoms, and may include bridged rings, fused rings, either heterocyclic, cyclic, or aryl rings.

The term “aryl” herein refers to aromatic cyclic compounds having up to 10 atoms, including carbon atoms, oxygen atoms, sulfur atoms, selenium atoms, etc. Aryl groups include, but are not limited to, groups such as phenyl, substituted phenyl, naphthyl, substituted naphthyl, in which the substituent is preferably lower alkyl, halogen, or lower alkyl. “Aryl” may also refer to fused rings systems having aromatic unsaturation. The fused ring systems can contain up to about 7 rings.

An “aryl group substituent” as used herein includes alkyl, cycloalkyl, cycloaryl, aryl, heteroaryl, optionally substituted with 1 or more, preferably 1 to 3, substituents selected from halo, haloalkyl, and alkyl, arylalkyl, heteroarylalkyl, alkenyl containing 1 to 2 double bonds, alkynyl containing 1 to 2 triple bonds, halo, hydroxy, polyhaloalkyl, preferably trifluoromethyl, formyl, alkylcarbonyl, arylcarbonyl, optionally substituted with 1 or more, preferably 1 to 3, substituents selected from halo, haloalkyl, alkyl, heteroarylcarbonyl, carboxyl, alkoxycarbonyl, aryloxycarbonyl, aminocarbonyl, alkylaminocarbonyl, dialkylaminocarbonyl, arylalkylaminocarbonyl, alkoxy, aryloxy, perfluoroalkoxy, alkenyloxy, alkynyloxy, arylalkoxy, aminoalkyl, alkylaminoalkyl, dialkylaminoalkyl, arylaminoalkyl, amino, alkylamino, dialkylamino, arylamino, alkylarylamino, alkylcarbonylamino, arylcarbonylamino, amido, nitro, mercapto, alkylthio, arylthio, perfluoroalkylthio, thiocyano, isothiocyano, alkylsufinyl, alkylsulfonyl, arylsulfinyl, arylsulfonyl, aminosulfonyl, alkylaminosulfinyl, dialkylaminosulfonyl, and arylaminosulfonyl.

The term “arylalkyl” as used herein refers to an alkyl group which is substituted with one or more aryl groups. Examples of arylalkyl groups include benzyl, 9-fluorenylmethyl, naphthylmethyl, diphenylmethyl, and triphenylmethyl.

The term “heteroaryl” for purposes of the present application refers to a monocyclic or multicyclic ring system, preferably about 5 to about 15 members, in which at least one atom, preferably 1 to 3 atoms, is a heteroatom, that is, an element other than carbon, including nitrogen, oxygen, or sulfur atoms. The heteroaryl may be optionally substituted with one or more, preferably 1 to 3, aryl group substituents. Exemplary heteroaryl groups include, for example, furanyl, thienyl, pyridyl, pyrrolyl, N-methylpyrrolyl, quinolyinyl and isoquinolinyl. The nomenclature alkyl, alkoxy, carbonyl, etc, is used as is generally understood by those of skilled this art. As used herein, aryl refers to saturated carbon chains that contain one or more carbon atoms; the chains may be straight or branched or include cyclic portions or may be cyclic.

The term “halogen” or “halide” includes F, Cl, Br, and I. This can include pseudohalides, which are anions that behave substantially similarly to halides. These compounds can be used in the same manner and treated in the same manner as halides. Pseudohalides include, but are not limited to, cyanide, cyanate, thiocyanate, selenocyanate, trifluoromethyl, and azide.

The term “haloalkyl” refers to a lower alkyl radical in which one or more of the hydrogen atoms are replaced by halogen, including but not limited to, chloromethyl, trifluoromethyl, 1-chloro-2-fluoroethyl, and the like. “Haloalkoxy” refers to RO— in which R is a haloalkyl group.

The term “sulfinyl” refers to —S(O)—. “sulfonyl” refers to —S(O)₂—.

“Aminocarbonyl” refers to —C(O)NH₂.

“Alkylene” refers to a straight, branched, or cyclic, preferably straight or branched, bivalent aliphatic hydrocarbon group, preferably having from 1 to about 20 carbon atoms. The alkylene group is optionally substituted with one or more alkyl group substituents. There may be optionally inserted along the alkylene group one or more oxygen, sulfur, or substituted or unsubstituted nitrogen atoms, wherein the nitrogen substituent is alkyl. Exemplary alkylene groups include methylene, ethylene, propylene, cyclohexylene, methylenedioxy, and ethylenedioxy. The term “lower alkylene” refers to alkylene groups having from 1 to 6 carbon atoms. Preferred alkylene groups are lower alkylene, with alkylene of 1 to 3 atoms being particularly preferred.

As used herein, when any particular group, such as phenyl or pyridyl, is specified, this means that the group is substituted or unsubstituted. Preferred substituents, where not specified, are halo, halo lower alkyl, and lower alkyl.

The term “library” refers to a collection of diverse compounds, in the present case, which are trisubstituted triazines.

The reaction can be conducted in any suitable solvent, including, but not limited to, DMXO, DMF, dioxane, water, ethanol, methanol, ethyl acetate, and the like. Exogenous heat energy, such as microwave energy, is applied to the system for about 1 to about 60 minutes to form styryl-based fluorescent dyes other types of energy which can be used to heat the system can be used, such as infrared energy, a heat source, or the like.

A library of 1152 compounds was synthesized and screened for promotion and inhibition activity. Screening for compounds that stimulate or inhibit pigmentation was performed in 24-well plates. Library compounds stored in 96-well plates in 10 mM DMSO were pipetted into wells preseeded with a fixed number (approximately 1,000,000) of melan-a immortalized melanocytes at a final concentration of 5-10 micromolar. For each set of plates tested, in addition to a vehicle control, a known pigment inhibitor, phenylthiourea (PTU, a tyrosinase inhibitor), was tested at 300 micromolar, and a known stimulator, isobutylmethylxanthine (IBMX), at 100 micromolar. The cells were cultured for 72 hours, and examined visually for any wells with cytotoxicity. Medium was removed, the cells were solubilized by the addition of 0.1 ml of 1 M NaOH in 10% DMSO for one hour, and transferred to 96 well plates. The melanin content was determined by a plate reader as OD450 after subtraction of a blank. The data are compared to untreated cells. Compounds causing >25% visually examined cytotoxicity at the tested concentrations were deemed to be inevaluable and were not scored. The results are shown in Table 1.

| | | % Change in pig- |
|---|---|---|
| Structures of Promoters | Concentration | mentation compared to untreated cells |
| 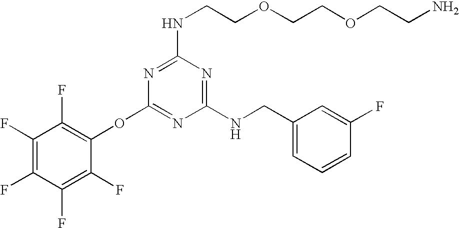 | 5 μM | 423 |
| 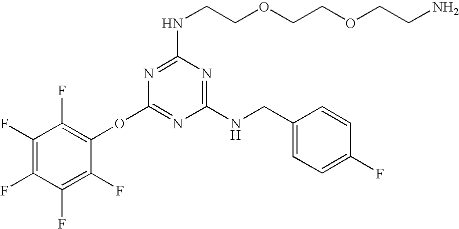 | 5 μM | 373 |
| 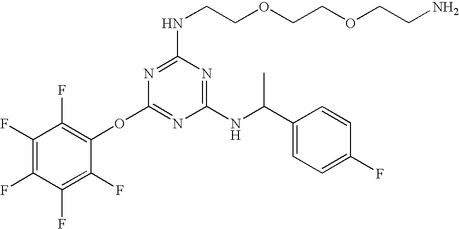 | 5 μM | 537 |
| 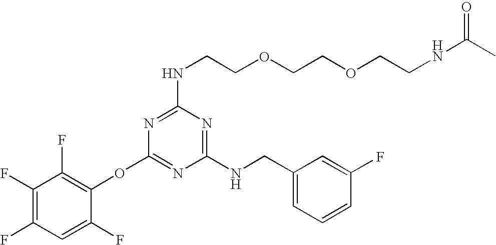 | 5 μM | 530 |
| 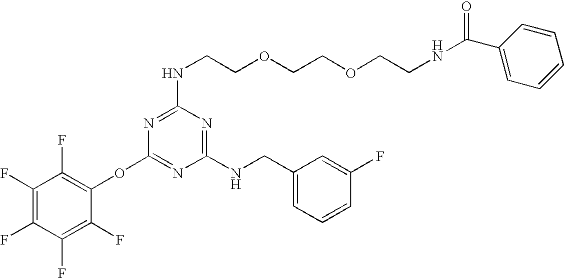 | 5 μM | 493 |
| 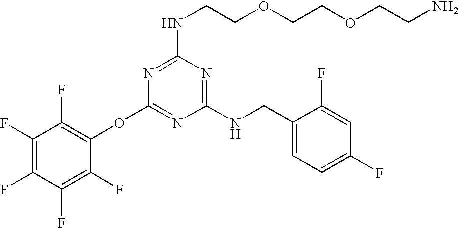 | 10 μM | 400 |
| 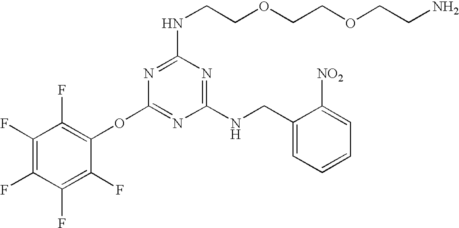 | 10 μM 3 | 360 |
| 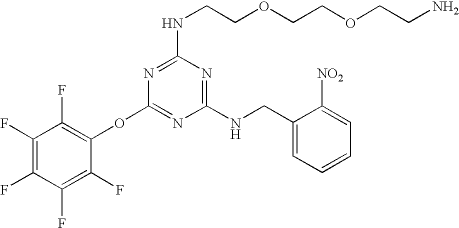 | 10 μM | 360 |
| 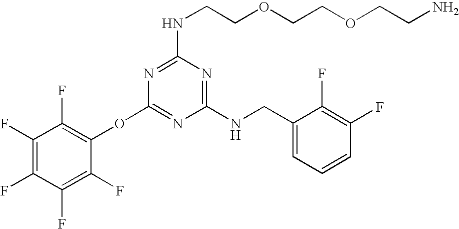 | 10 μM | 353 |
| 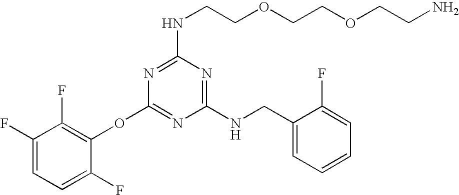 | 5 μM | 277 |
| 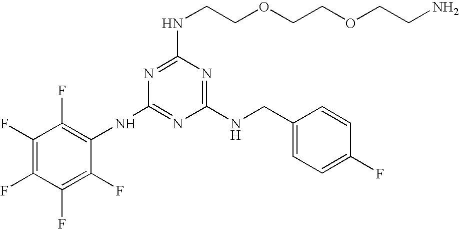 | 5 μM | 270 |
| 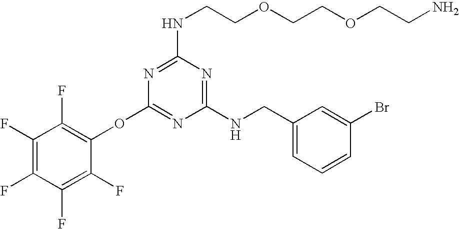 | 5 μM | 270 |
| 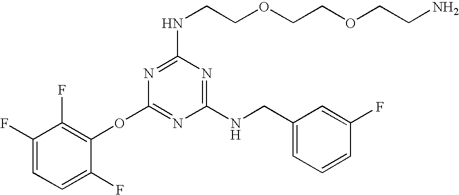 | 5 μM | 227 |
| 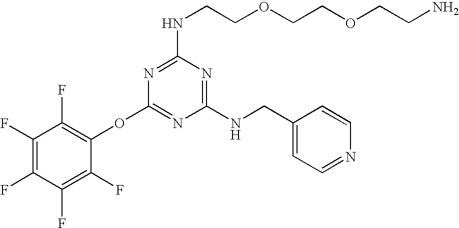 | 5 μM | 227 |
| 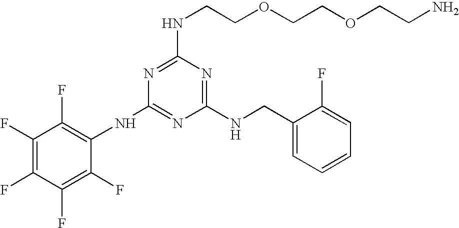 | 5 μM | 170 |
| 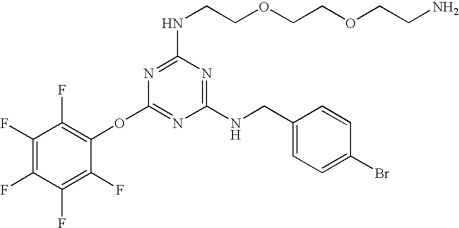 | 1 μM | 167 |
| 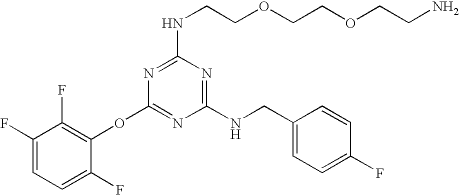 | 5 μM | 160 |
| 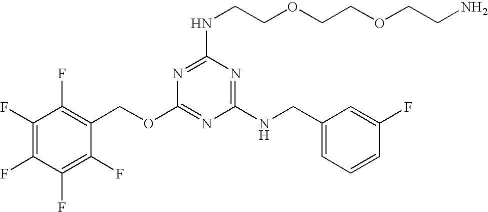 | 10 μM | 102 |
| 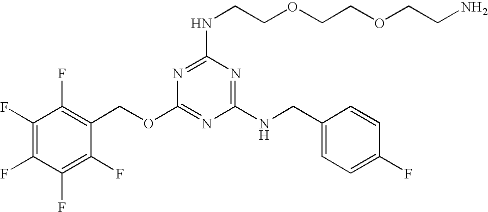 | 10 μM | 87 |
| 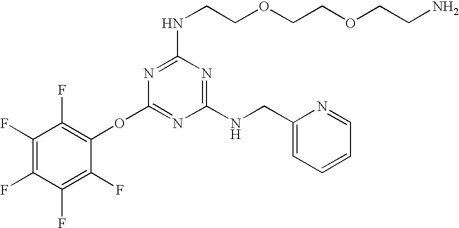 | 5 μM | 72 |
| 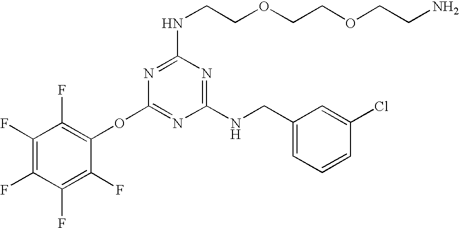 | 5 μM | 109 |
| 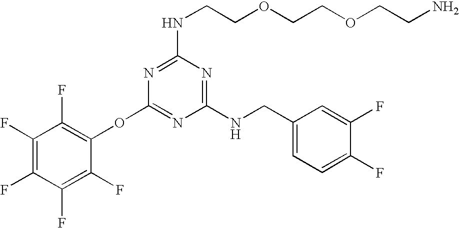 | 5 μM | 103 |
| 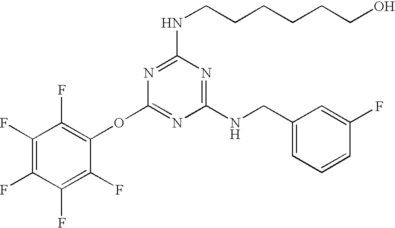 | 5 μM | 182 |
| 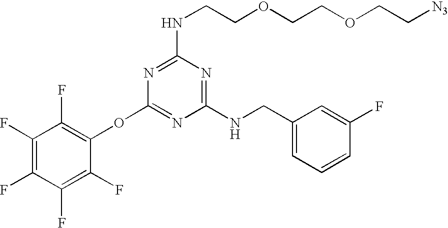 | 5 μM | 197 |
| 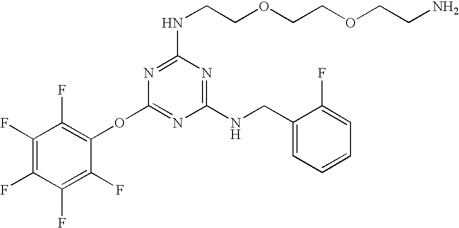 | 5 μM | 340 |
| 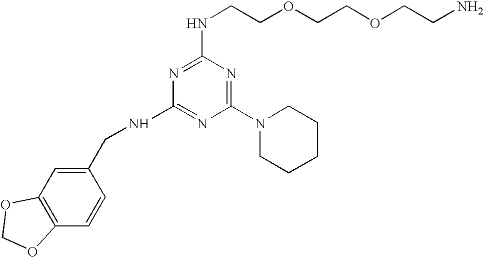 | 10 μM | 20 |
| 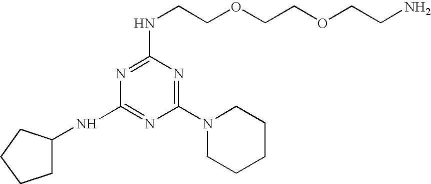 | 10 μM | 33 |
| 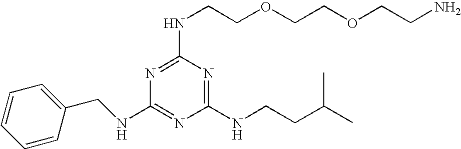 | 10 μM | 35 |
| 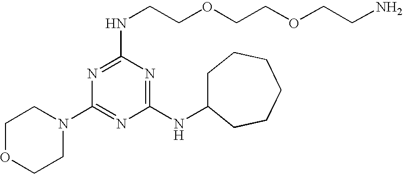 | 10 μM | 50 |
| 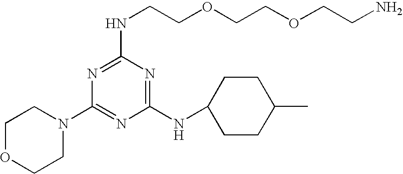 | 10 μM | 55 |
| 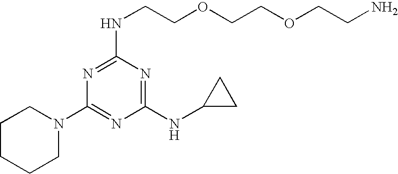 | 10 μM | 53 |
| 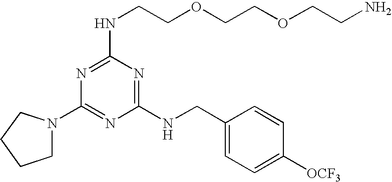 | 10 μM | 57 |
| 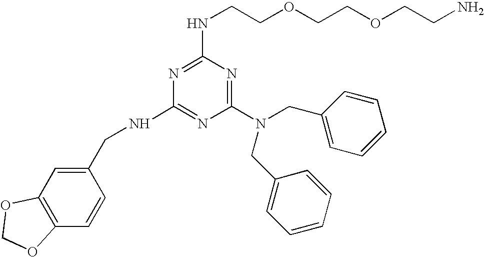 | 10 μM | 50 |
| 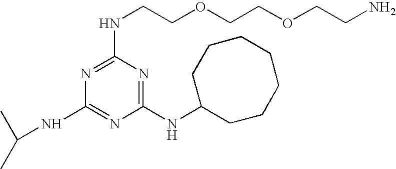 | 10 μM | 49 |
| 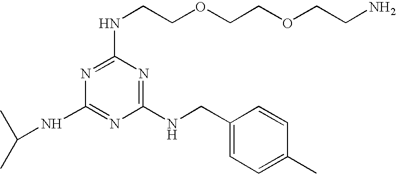 | 10 μM | 46 |
| 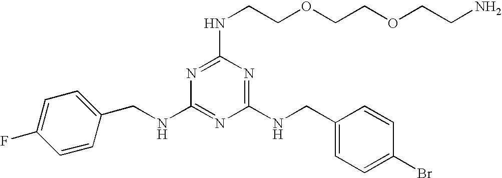 | 10 μM | 44 |
| 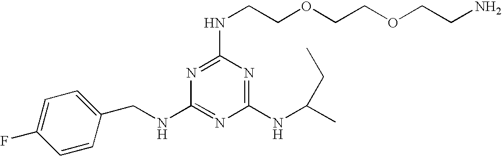 | 10 μM | 55 |
| 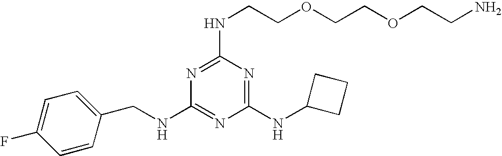 | 10 μM | 40 |
| 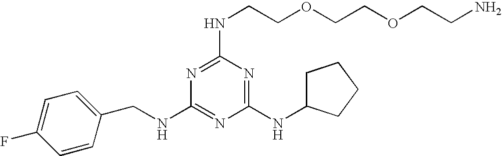 | 10 μM | 52 |
| 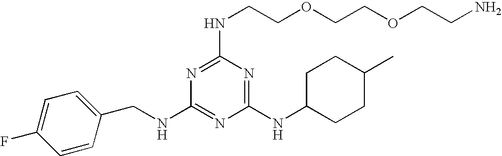 | 10 μM | 62 |
| 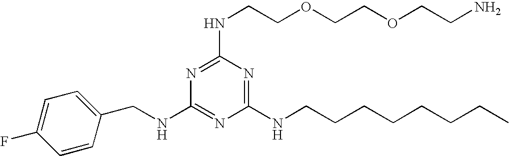 | 10 μM | 48 |
| 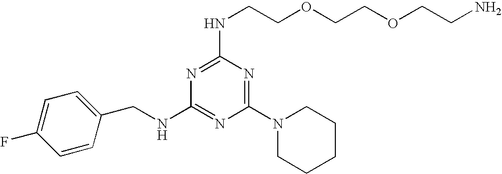 | 10 μM | 41 |
| 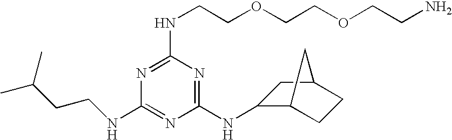 | 10 μM | 52 |
| 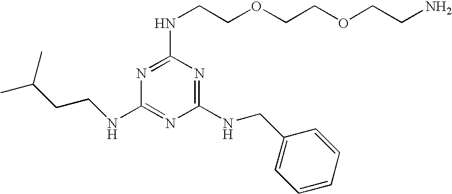 | 10 μM | 69 |
| 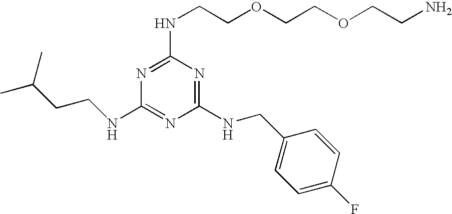 | 10 μM | 37 |
| 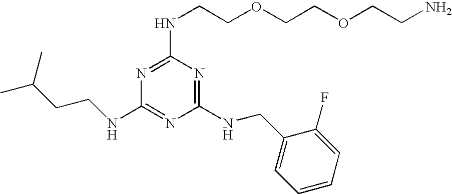 | 10 μM | 37 |
| 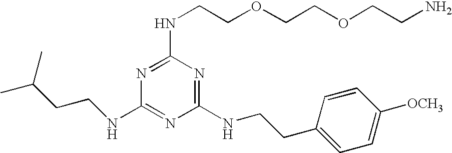 | 10 μM | 41 |
| 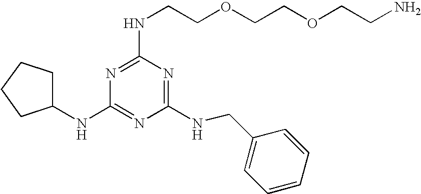 | 10 μM | 48 |
| 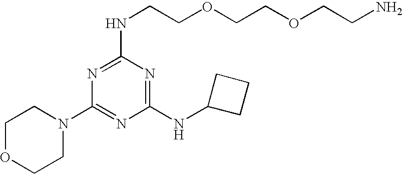 | 10 μM | 52 |
| 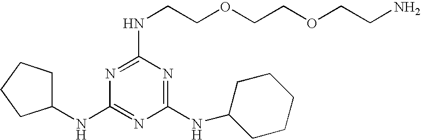 | 10 μM | 38 |
| 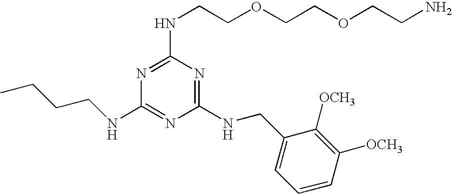 | 10 μM | 52 |
| 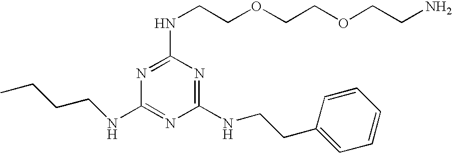 | 10 μM | 48 |
| 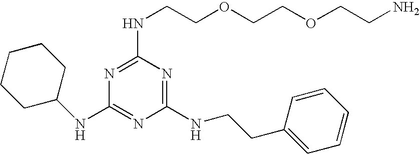 | 10 μM | 44 |
| 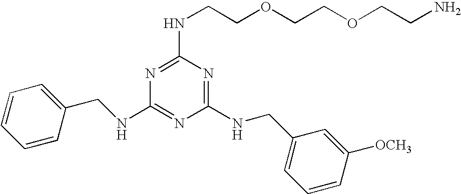 | 10 μM | 53 |
| 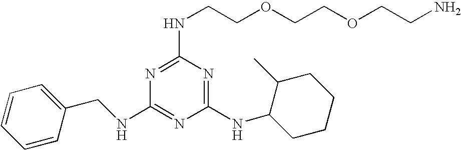 | 10 μM | 53 |
| 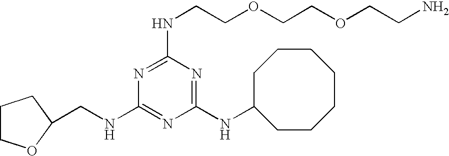 | 10 μM | 51 |
| 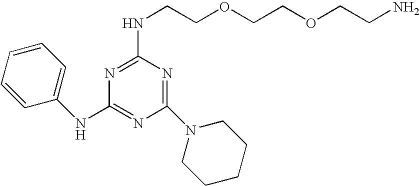 | 10 μM | 43 |
| 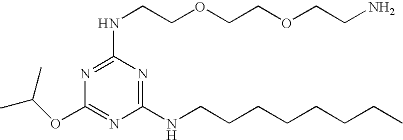 | 10 μM | 41 |
| 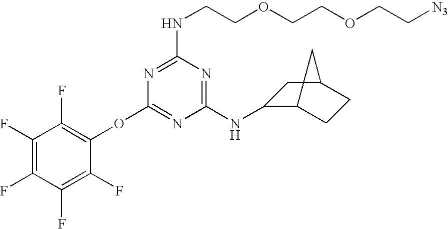 | 10 μM | 54 |
| 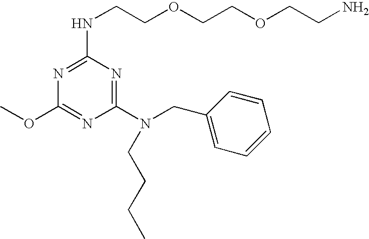 | 10 μM | 60 |

The active molecules may be readily used to create affinity matrices such as agarose or sepharose beads for protein target isolation.

In a similar fashion, mitochondrial ATPase can be used to screen for compounds that modulate pigmentation. This screening can be effected with ATPase per se, or with mRNA for ATPase, or protein or activity to screen for compounds that modulate pigmentation.

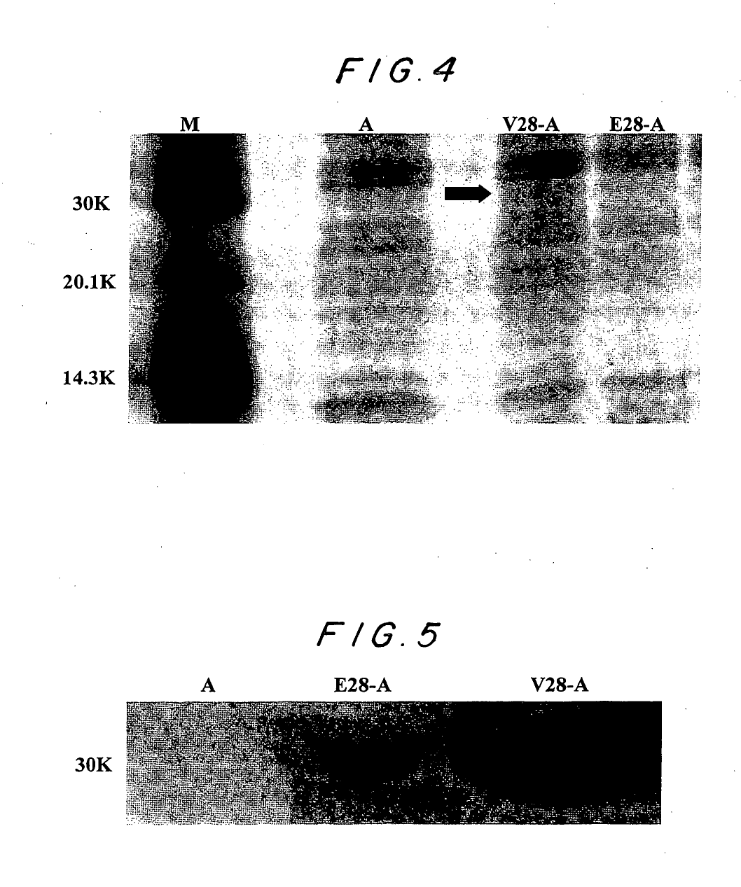

As shown in FIG. 4, mitochondrial ATPase is the target of PPA, using affinity chromatography using immobilized PPA (PPA-Affi). Melanocyte extracts were incubated with PPA-Affi in the presence of the compounds shown. After extensive washing, bound proteins were then eluted with SDS-containing buffer and subjected to SDS-PAGE and silver staining. Subunits of the mitochondrial F₁F₀ ATPase specifically bound to an eluted from the PPA-Affi, but not matrix, reacted with ethanolamine rather than PPA. The binding of mitochondrial ATPase was inhibited not only by PPA itself but also by oligomycin, a known mitochondrial ATPase inhibitor.

FIG. 5 is another illustration that mitochondrial ATPase is the target of PPA. The experimental protocol were similar to those described above, but rather than silver stain, immunoblotting was conducted with antibodies to the beta and alpha subunits of the mitochondrial ATPase. The primary antibody in A was anti-F₁-ATPase beta subunit antibody (1:2500, 1 hour), and the primary antibody in B was anti-ATPase alpha subunit antibody (1:2500, 1 hour). The secondary antibody was anti-mouse Goat IgG (1:2500, 1 hour). PPA, ADA, and oligomycin all competed with ATPase binding to a PPA affinity matrix.

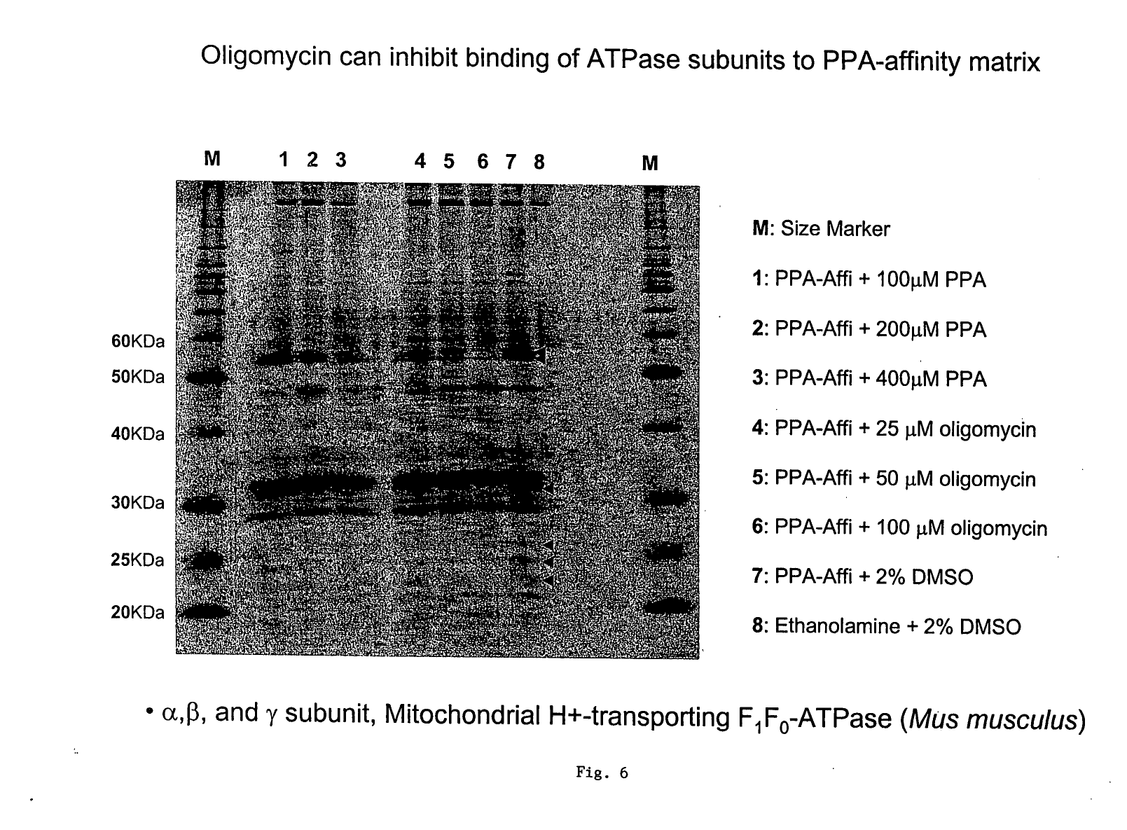

FIG. 6 illustrates that PPA6 and aurovertin do not compete for binding of ATPase to PPA-Affi. For this experiment, the cell source was Melan-a (murine melanocyte). The reaction volume was 147 microL of protein extract (0.3 mg/mL)+147 microL bead buffer+6 microL competitor (aurovertin or PPA6 or DMSO). The competition took place for 30 minutes at 4° C. with rocking. The bead volume was 40 microL of 50% slurry. The beads were washed with 1 mL bead buffer seven times. Elution was with 40 microL+30 microL Laemmli buffer, heating for 4 minutes at 90° C.

For the gel electrophoresis, the gel was 10% SDS-PAGE gel, 20-well combination. The loading volume was 25 microL (2^{nd} elution). Running was 30V, overnight.

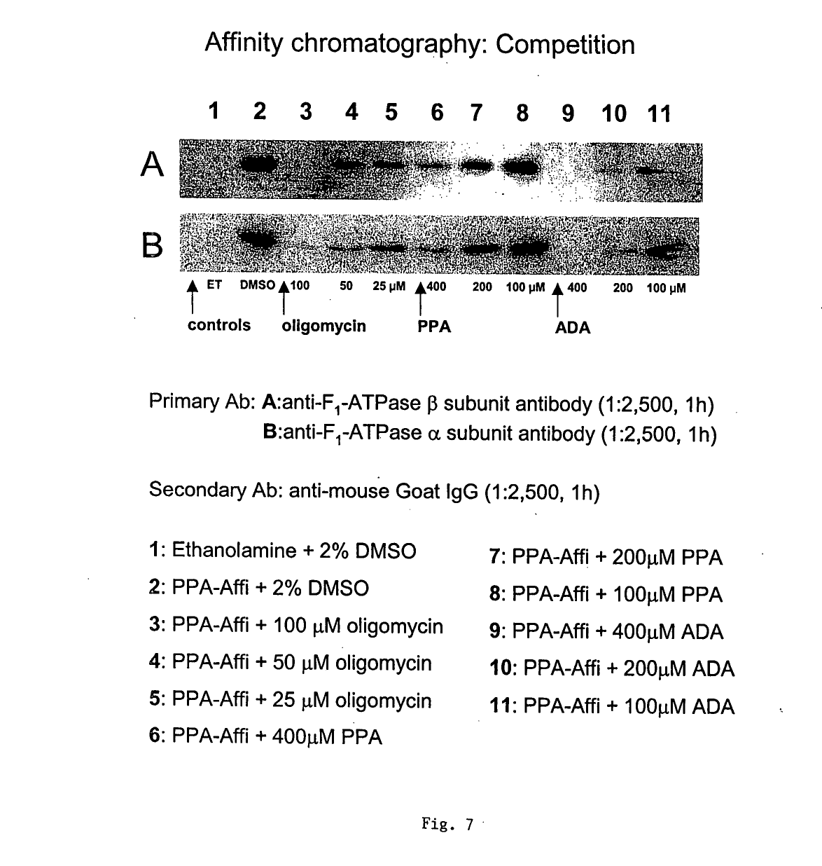

FIG. 7 shows that similar results are obtained with human melanocytic cell line (SK Mel19). Using human melanoma cell source SK Mel 19, the reaction volume was 147 microL of protein extract (1.0 mg/mL)+147 microL bead buffer+6 microL competitor (oligomycin) or S+DMSO. Competition was effected for 30 minutes at 4° C. with rocking. The bead volume was 40 microL of 50% slurry. Washing was five times with 1 mL bead buffer, and elution was with 40 microL Laemmli buffer, with heating for 4 minutes at 90° C. Gel electrophoresis was with 10% SDS-PAGE gel, 20 well combination. Loading volume was 35 microL, and running was 30V, overnight. Here, one can see that both the beta subunit and OSCP (oligomycin sensitivity conferring protein) are specifically bound to a PPA-Affi matrix and are eluted by oligomycin.

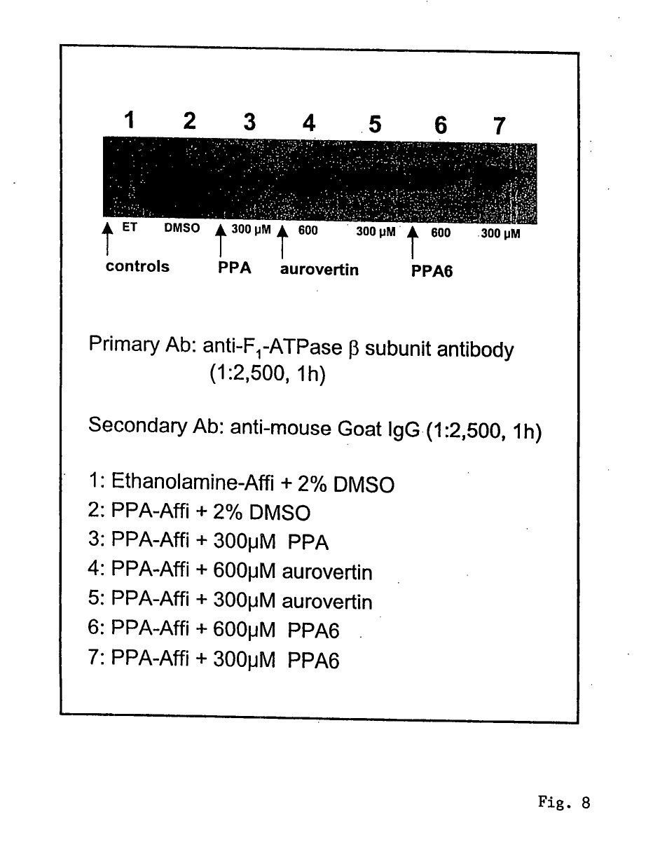

FIG. 8 shows the SK Mel 19 with an antibody to the subunit of ATPase, illustrating the same results as in FIG. 7. Using human melanoma cell source SK Mel 19, the reaction volume was 99 microL of protein extract (1.0 mg/mL)+99 microL bead buffer+2 microL competitor (oligomycin) or DMSO. Incubation was effected for 30 minutes at 4° C. with rocking. The bead volume was 40 microL of 50% slurry. Washing was five times with 1 mL bead buffer, and elution was with 40 microL Laemmli buffer, with heating for 4 minutes at 90° C. Gel electrophoresis was with 10% SDS-PAGE gel, 20 well combination. Loading volume was 35 microL, and running was 100V for one hour.

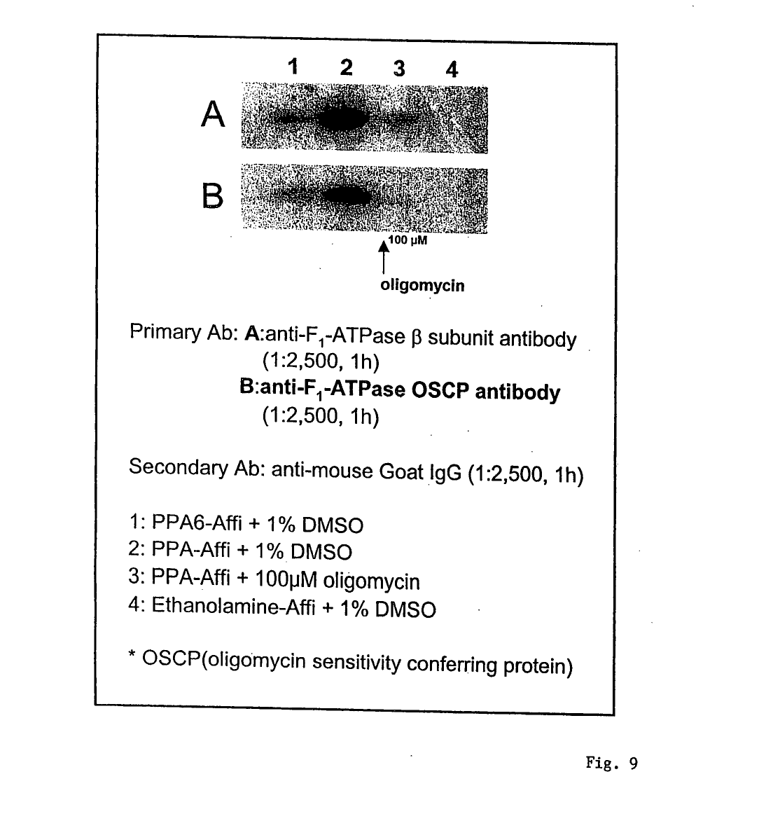

FIG. 9 illustrates that PPA, oligomycin, and aurovertin all promote pigmentation.

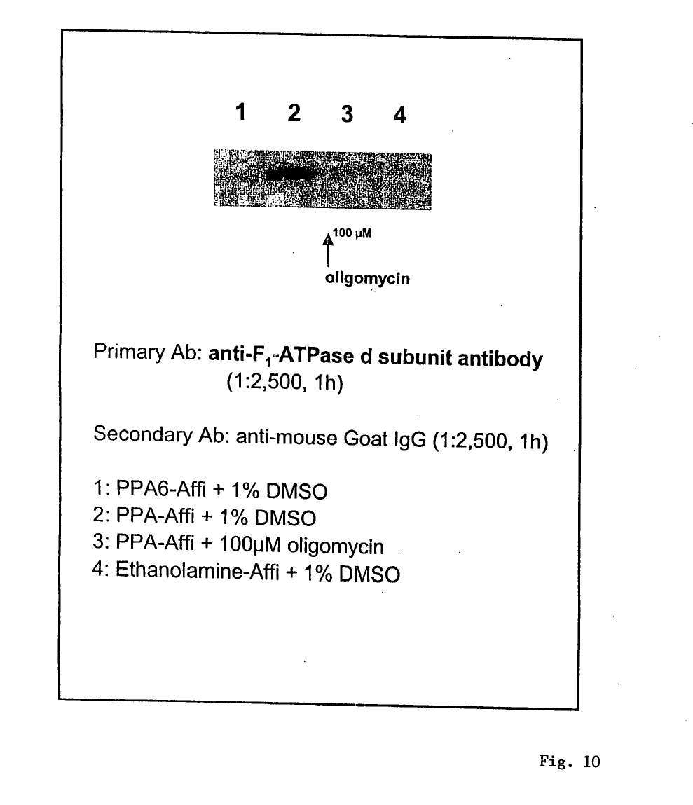

FIG. 10 shows that both PPA and oligomycin promote pigmentation.

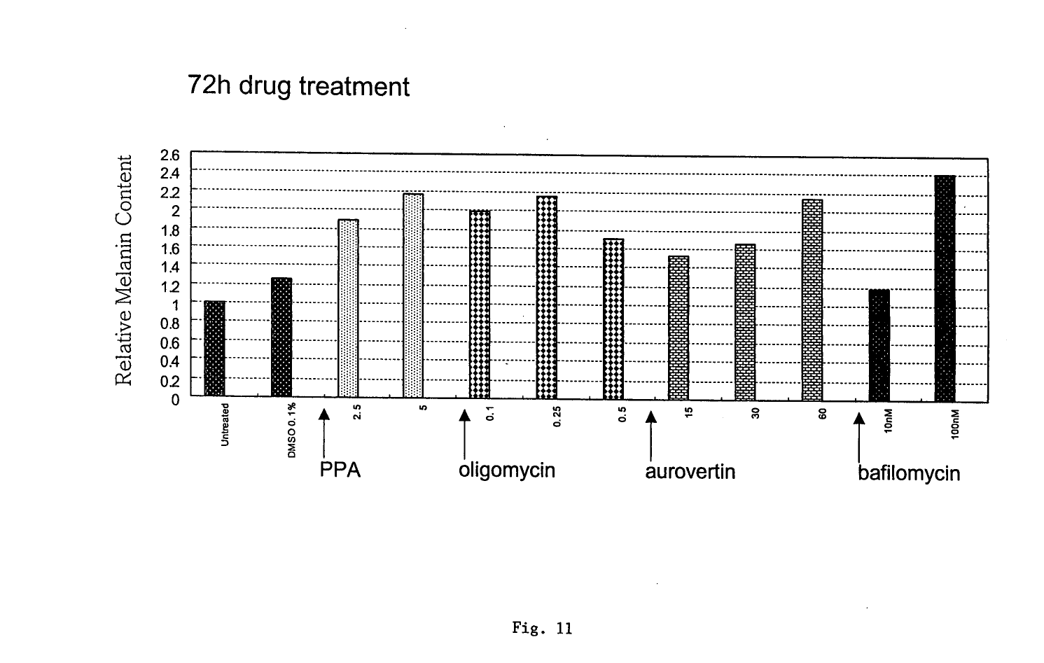

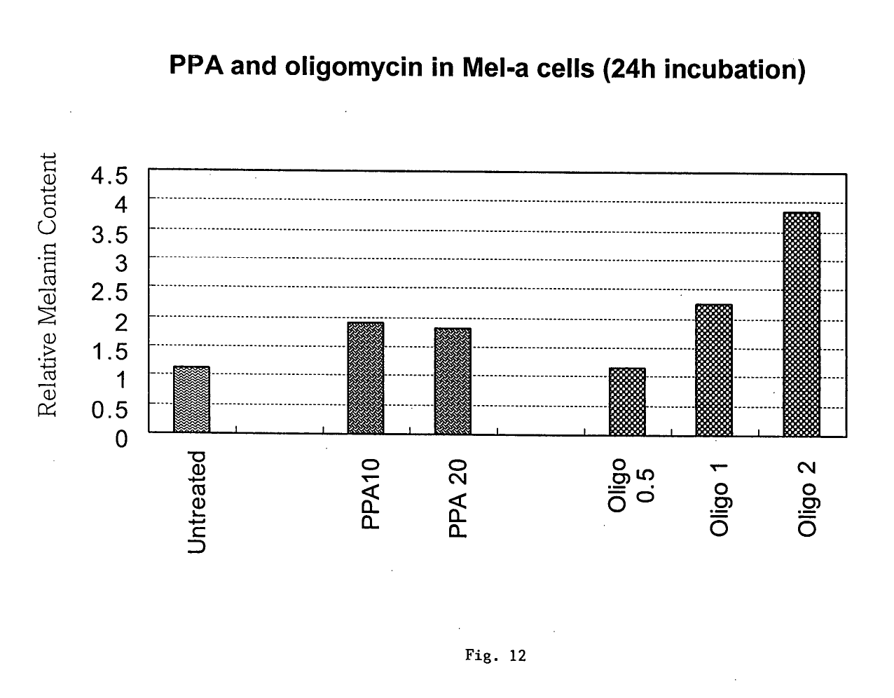

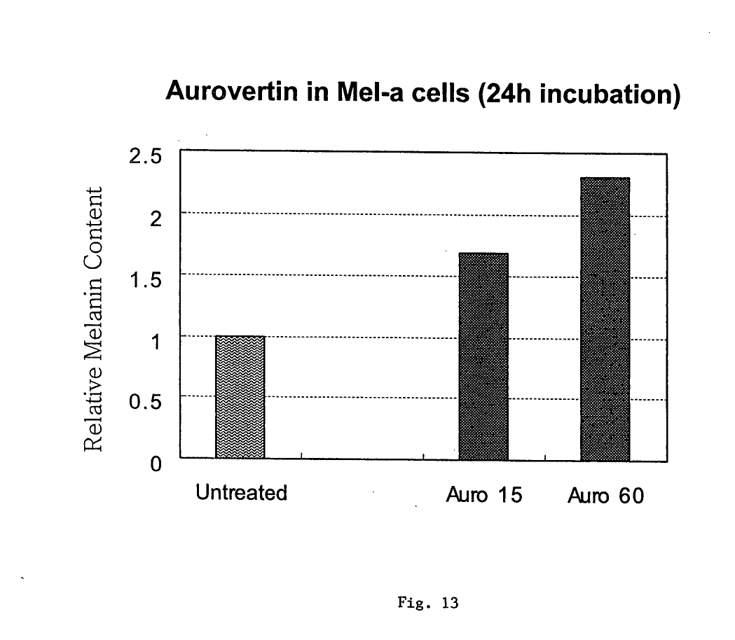

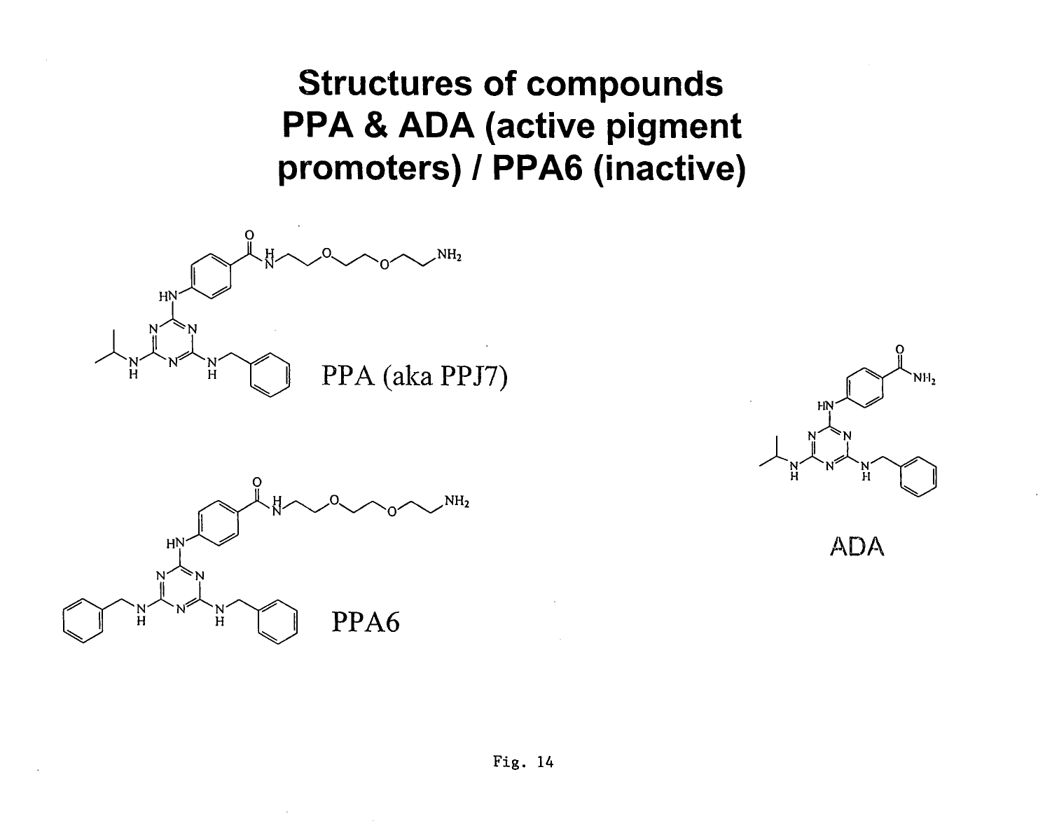

FIG. 11 shows that aurovertin promotes pigmentation.

Melanin Assay

The cells were rinsed with phosphate buffered saline (PBS) and lysed with an extraction buffer of 50 nM Tris, pH 7.5; 2 mM ethylenediamine tetraacetic acid (EDTA), pH 7.8; 150 mM NaCl; 1% Titron X-100, with protease inhibitor cocktail at 4° C. The cell extracts were then spun at 12,000 rpm for 10 minutes at 4° C. The remaining pellet was assayed for melanin by rinsing twice with ethanol-ether (1:1) and dissolving in 200 microL of 2N NaOH in 20% DMSO at 60° C. A 100 microL aliquot of the resulting solution was then measured for absorbance at 490 mm.

Isolation and Identification of Drug Cellular Target(s)

Protein was extracted from cells by incubation with extraction buffer (1 mM CaCl2, 150 mM NaCL; 10 mM Tris, pH 7.4; 1% Triton X-100, 1 mM PMSF plus protease inhibitor cocktail (Roche, NJ, USA) for five minutes on ice. Crude lysate was centrifuged at 13,000 rpm for ten minutes. The protein concentration of the supernatant was measured by the Bradford assay (Bio-Rad, CA) and adjusted to a final concentration of 1 microg/microL prior to affinity chromatography.

25-20 microL of agarose affinity matrix conjugated compound was washed with 1 mL bead buffer (10 mM Tris, pH 7.4; 5 mM NaF; 250 mM NaCl; 5 mM EDTA; 5 mM EGTA; 0.1% Triton X-100 plus protease inhibitor cocktail (Roche, NJ). Matrices were incubated with 50-200 microg of protein extract plus an identical volume of bead buffer at 4° C. or 30° C. For studies of competition between drug and cellular target, the competitor was added to the mixture of protein extract/bead buffer and incubated at 4° C. for 30 minutes prior to incubation with the matrix. The supernatant containing unbound proteins was removed by centrifugation and the matrices were washed seven times with 1 mL bead buffer. Proteins bound to the matrices were eluted by incubation with 50 microL Laemmli buffer (Bio-Rad, CA) at 94° C. for three minutes.

The total volume of the eluted proteins were separated by 7.5% or 10% SDS-PAGE and visualized by silver staining (Amersham, NJ). Prominent protein bands specific to active matrices were excised from each gel and identified by Ion Trap mass spectrometry (NYU Protein Analysis Facility, Skirball Institute of Bimolecular Medicine, NY).

Antibodies and Reagents

Monoclonal antibodies against the alpha, beta, delta, and OSCP subunits of the ATP synthase (F₁F₀) were purchased from Molecular Probes, Inc., Eugene, Oreg. Oligomycin and aurovertin B were purchased from Sigma-Aldrich, MO. Bafilomycin Al was purchased from Wako Pure chemical Industries. Lt., Osaka, Japan.

Western Blot Analysis

Proteins were separated by 7.5% or 10% SDS-PAGE and transferred onto membranes (Immobilon-P, Millipore,. Waltham, Mass.).

Thus, the present invention provides novel methods of screening for compounds that inhibit or stimulate melanogenesis. Compounds identified using the methods of the present invention are useful for treating diseases and cosmetic conditions associated with the underproduction or overproduction of melanin.

As noted above, compounds that interact with mitochondrial ATPase have been found to promote pigmentation. Accordingly, mitochondrial ATPase, ATPase mRNA, and related compounds can be used to screen for compounds that modulate pigmentation. As described supra, mitochondrial ATPase or antibodies thereto can be used to demonstrate whether a compound binds to ATPase or an antibody or protein thereof.

The compounds of the present invention which have been found to stimulate melanin production can be used topically for skin darkening, or to produce a safe tan. A target for the molecules can be identified and used as a screen to identify more new compounds which alter pigmentation.

Those compounds which are found to inhibit pigmentation can be used as topical agents for skin or hair lightening. Additionally, a target for these molecules can be identified and used as a screen to identify more new compounds that alter pigmentation.

The compounds of the present invention which have been found to stimulate melanin production can be used in conjunction with cAMP elevating agents and/or MAP kinase inhibitors, which act synergistically with the trisubstituted triazines to increase pigmentation. Additionally, protein kinase C inhibitors can be used with the compounds described herein to augment pigmentation.

To inhibit melanin production, those trisubstituted triazines which have been found to inhibit melanin production can be used in conjunction with protein kinase A inhibitors to augment their ability to inhibit pigmentation.

The following table illustrates some of the agents that can be used in conjunction with the trisubstituted triazines or compounds that interact with mitochondrial ATPase of the present invention to enhance or to inhibit melanin production. Of course, this table does not limit the agents that can be used with the trisubstituted triazines or other compounds that interact with mitochondrial ATPase.

| | Chemical Name | Source |
|---|---|---|
| cAMP Elevating Agents | | |
| MSH | Melanocyte Stimulating Hormone | Sigma-Aldrich St. Louis, MO |
| IBMX | 3-Isobutyl-1-methylxanthine | Sigma-Aldrich St. Louis, MO |
| Forskolin | Forskolin | Sigma-Aldrich St. Louis, MO |
| Cholera Toxin | Vibrio cholerae toxin | Calbiochem-San Diego, CA |
| Prostaglandin E2 | Prostaglandin E2 | Calbiochem-San Diego, CA |
| 8-Br-cAMP | 8-Br-cAMP | Calbiochem-San Diego, CA |
| Protein Kinase A | | |
| Inhibitors | | |
| H89 | N-[2-(p-Bromocinnamylamino)ethyl]-5-isoquinolinesulfonamide-2HCl | BIOMOL-Plymouth Meeting, PA |
| KT5720 | KT5720 | Calbiochem-San Diego, CA |
| PKA inhibitor peptide | PKA inhibitor peptide | Upstate-Charlottesville, VA |
| PKA inhibitor cocktail | PKA inhibitor cocktail | Upstate-Charlottesville, VA |
| Rp-cAMP | Rp-cAMP | Calbiochem-San Diego, CA |
| Rp-8-CPT-cAMPS | Rp-8-CPT-cAMPS | Sigma-Aldrich St. Louis, MO |
| 5-24 | (H-TTYADFIASGRTGRRNAIHD) peptide inhibitor | Calbiochem-San Diego, CA |
| 14-22 | (Myr-GRTGRRNAI-NH2) heat-stable peptide inhibitor | Calbiochem-San Diego, CA |
| Protein Kinase C | | |
| Inhibitors | | |
| Ro 31-8220 | Ro 31-8220 | BIOMOL-Plymouth Meeting, PA |
| 19-27 | (Myristoyl-Phe-Ala-Arg-Lys-Gly-Ala-Leu-Arg-Gln) peptide inhibitor | Calbiochem-San Diego, CA |
| GF109203X | (2-[1-(3-Dimethylaminopropyl)-1H-indol-3-yl]-3-(1H-indol-3-yl)-maleimide | Calbiochem-San Diego, CA |
| Rottlerin | Mallotoxin | Calbiochem-San Diego, CA |
| MAPK Inhibitors | | |
| PD98059 | PD98059 | BIOMOL-Plymouth Meeting, PA |
| PD184352 | PD184352 | Pfizer-Ann Arbor, Michigan |
| U0126 | (1,4-Diamino-2,3-dicyano-1,4bis(2-aminophenylthio)butadiene) | Calbiochem-San Diego, CA |
| SB202190 | SB202190 | Upstate-Charlottesville, VA |
| CNI-1493 | CNI-1493 | Cytokine PharmaSciences-King of Prussia, PA |

Once a compound has been identified as one that strongly inhibits pigmentation or as one that strongly stimulates pigmentation, libraries of additional similar compounds can be screened for their ability to stimulate or inhibit pigmentation. Once a compound is found to inhibit or stimulate pigmentation, molecular modeling techniques can be used to design chemical analogs of the compound that are as effective or are even more effective. For example, chemical analogs of V28 or V30 can be created using these or other modeling techniques.

Examples of molecular modeling systems are the CHARM (Polygen Corporation, Waltham, Mass.) and QUANTA (Molecular Simulations, Inc., San Diego, Calif.) programs. CHARM performs the energy minimization and molecular dynamics functions. Quanta performs the construction, graphic modeling, and analysis of molecular structure. Quanta allows interactive constructions, modification, visualization, and analysis of the behavior of molecules with each other.

Once a compound that affects or inhibits pigmentation is identified, the compound can be used to generate a hypothesis. Such a hypothesis can be generated from any one of the preferred compounds of the present invention using, e.g., the program CATALYST (Molecular Simulations, Inc., San Diego, Calif.). Furthermore, CATALYST can use the hypothesis to search proprietary database such as, for example the Cambridge small molecule database (Cambridge, England), as well as other databases or compound libraries, e.g., those cited above, to identify additional examples of compounds of the present invention.

Compounds of the present invention can further be used to design more effective analogs using modeling packages such as Ludi, Insight, C2-Minimizer, and Affinity (Molecular Simulations, Inc., San Diego, Calif.). A particularly preferred modeling package is MacroModel (Columbia University, New York, N.Y.).

Experiments were conducted to illustrate protein binding of specific compounds. One mg of melan-a (a wild-type mouse melanocyte) cell lysate was incubated with 25 microliters of the packed affinity matrices for 30 minutes at 37° C. The results are shown in FIG. 4.

In FIG. 4, lane “M” corresponds to the molecular weight markers and “A” denotes the unconjugated ethanolamine treated agarose bead matrix. “V28-A” and “E28-A” represent the corresponding TG library molecules linked to agarose bead affinity matrices. E28 is an inactive molecule identified in in vitro screens which is structurally similar to the active V28 compound. The 30 kilodalton band is present in the V28-A lane but not in the A or E28-A lanes. The band was excised and sequenced using MS/MS-QTOF analysis. The amino acid sequence results identified the band as prohibitin.

Western blot analysis of melan-a (wild-type mouse melanocyte) cell lysate was incubated with 25 microliters of packed affinity matrix for 30 minutes at 37° C. In FIG. 5, “A” denotes the unconjugated ethanolamine treated agarose bead matrix. “V28-A” and “E28-A” represent the corresponding TG library molecules linked to agarose bead affinity matrices. The 30-K protein band corresponding to prohibitin. Proteins were resolved on a 5% to 15% SDS-PAGE gel before transfer to a PVDF membrane. The mouse monoclonal anti-prohibitin antibody was purchased from LabVision/Neomarkers (Fremont, Calif.) and used at a 1:50 dilution.

Thus, prohibitin or molecules that interact with prohibitin can be used to screen for pigment stimulators or inhibitors. Compounds that interact with prohibitin or that alter levels of prohibitin mRNA or protein can be used to alter skin pigmentation.

The compounds of the present invention can be used to treat animals, particularly humans, that have diseases, conditions, or disorders caused by or associated with the production or overproduction of melanin. These diseases, conditions, or disorders include those that can be characterized by discolorations of the skin or hair such as, for example, hyperpigmentation caused by inflammation or from diseases such as melasma, or brown spots such as “café au lait” macules. Alternatively, a subject may wish to use a pigmentation inhibitor to lighten or a pigmentation promoter to stimulate the color of his or her hair or skin.

Thus, the compounds of the present invention are useful in treating disorders of human pigmentation, including solar and simple lentigines (including age/liver spots), melasma/chloasma and postinflammatory hyperpigmentation. These compounds reduce melanin levels in the skin by inhibiting the production of melanin, whether the melanin is produced constitutively or in response to ultraviolet radiation, such as sun exposure. Thus, some of the active compounds in the present invention can be used to reduce skin melanin content in non-pathological states so as to induce a lighter skin tone, as desired by the user, or to prevent melanin accumulation in the skin that has been exposed to ultraviolet radiation. These compounds can also be used in combination with skin peeling agents, including glycolic acid or trichloroacetic acid face peels, to lighten skin tone and to prevent repigmentation.

Other compounds useful according to the present invention are useful in treating skin conditions where insufficient skin pigmentation is produced, or where the subject, for cosmetic purposes, simply wishes to develop a “sunless tan” or to augment tanning induced by a limited exposure to sunlight or ultraviolet light. Unlike previously known indoor tanning compositions, the compounds of the present invention actually produce additional melanin in the skin, and thus protect the skin from ultraviolet radiation. The appropriate dose regimen, the amount of each dose administered, and specific intervals between doses of the active compound will depend upon the particular active compound employed, the condition of the patient being treated, and the nature and severity of the disorder or condition being treated. Preferably, the active compound is administered in an amount and at an interval that results in the desired treatment of or improvement in the disorder or condition being treated.

For skin lightening, an active compound of the present invention can also be used in combination with sun screens (for example, UVA and/or UVB blockers) to prevent repigmentation, to protect against sun or UV-induced skin darkening or burning, or to enhance their ability to reduce skin melanin and their skin bleaching action. For skin lightening, an active compound used in the present invention can also be used in combination with retinoic acid or its derivatives or any compounds that interact with retinoic acid receptors and accelerate or enhance the invention's ability to reduce skin melanin, or to enhance the invention's ability to prevent the accumulation of skin melanin.

For skin lightening, an active compound used in the present invention can also be used in combination with 4-hydroxyanisole. For skin lightening, the active compounds used in this invention can also be used in combination with ascorbic acid, its derivatives, and ascorbic acid-based products, such as magnesium sorbate, or other products with an anti-oxidant mechanism such as resveratrol which accelerate or enhance their ability to reduce skin melanin and their skin bleaching action.

Tricyclic antidepressants are other preferred hydrophobic amines that can be used in the methods and compositions of the present invention. Particularly preferred tricyclic antidepressants include imipramine, nortriptyline, protriptyline, trimipramine, and doxepin. Another compound useful in the methods and compositions of the invention is sphingosine, which is also commercially available.

As one skilled in the art will readily appreciate, the compounds of the present invention can be used alone or in combination with each other, as well as in combination with the other melanin inhibiting and melanin stimulating compounds described above, and with any other pigment affecting compounds.

Thus, the present invention relates both to methods for modulating the pigmentation of skin in which the active compound used according to the invention and one or more other active ingredients referred to above, are administered together, as part of the same pharmaceutical composition, as well as methods in which they are administered separately as part of an appropriate dose regimen designed to obtain the benefits of the combination therapy. The appropriate dose regimen, the amount of each dose administered, and specific intervals between doses of each active agent will depend upon the specific combination of active agents employed, the condition of the patient being treated, and the nature and severity of the disorder or condition being treated. Such additional active ingredients will generally be administered in amounts less than or equal to those for which they are effective as single topical therapeutic agents. The FDA approved dosages for such active agents that have received FDA approval for administration to human are publicly available.

For example, any of the compounds of the present invasion known to be inhibitors of pigmentation may be used in combination with a tyrosinase inhibitor or other skin-whitening agent as currently known in the art or to be developed in the future, including any one or more of agents described in patent publications as follows: U.S. Pat. No. 4,278,656 to Nagai et al., issued Jul. 14, 1981; U.S. Pat. No. 4,369,174 to Nagai et al., issued Jan. 18, 1983; U.S. Pat. No. 4,959,393 to Toriara et al., issued Sep. 25, 1990; U.S. Pat. No. 5,580,549 to Fukuda et al., issued Dec. 3, 1996; U.S. Pat. No. 6,123,959 to Jones et al., issued Sep. 26, 2000; U.S. Pat. No. 6,132,740 to Hu, issued Oct. 17, 2000; 30 U.S. Pat. No. 6,159,482 to Tuloup et al., issued Dec. 12, 2000; WO 99/32077 by L'Oreal, published Jul. 1, 1999; WO 99/64025 by Fytokem Prod. Inc., published Dec. 16, 1999; WO 00/56702 by Pfizer Inc., published Sep. 28, 2000; WO 00/76473 by Shiseido Co. Ltd., published Dec. 12, 2000; EP 9,971,140 by L'Oreal S A, published May 3, 2000; JP 5,221,846 by Kunimasa Tomoji, published Aug. 31, 1993; JP 7,242,687 by Shiseido Co. Ltd., published Sep. 19, 1995; JP 7,324,023 by Itogawa H. published Dec. 12, 1995; JP 8,012,552 by Shiseido Co. Ltd., published Jan. 16, 1996; JP 8,012,554 by Shiseido Co. Ltd., published Jan. 16, 1996; JP 8,012,557 by Shiseido Co. Ltd., published Jan. 16, 1996; JP 8,012,560 by Shiseido Co. Ltd., published Jan. 16, 1996; JP 8,012,561 by Shiseido Co. Ltd., published Jan. 16, 1996; JP 8,134,090 by Fujisawa, published May 28, 1996; JP 8,168,378 by 5 Kirinjo KK, published Jul. 2, 1996; JP 8,277,225 by Kansai Koso KK, published Oct. 22, 1996; JP 9,002,967 by Sanki Shoji KK, published Jan. 7, 1997; JP 9,295,927 by Yagi Akira, published Nov. 18, 1997; JP 10/072,330 by Kansai Kouso, published Mar. 17, 1998; JP 10/081,626 by Kamiyama KK, published Mar. 31, 1998; JP 10/101,543 by Kansai Kouso KK, published Apr. 21, 1998; JP 11/071,231 by Maruzen Pharm., published Mar. 16, 1999; JP 11/079,934 by Kyodo Nyugyo, published Mar. 23, 1999; JP 11/246,347 by Shiseido Co. Ltd., published Sep. 14, 1999; JP 11/246,344 by Shiseido Co. Ltd., published Sep. 14, 1999; JP 2000-080023 by Kanebo Ltd., published Mar. 21, 2000; JP 2000-095663 by Kose KK, published Apr. 4, 2000; JP 2000-159681 by Hal Tai Confectionary Co. Ltd., published Jun. 13, 2000; JP 2000-247907 by Kanebo Ltd., published Sep. 12, 2000; JP-9002967 15 by Sanki Shoji KK, published Jan. 7, 1997; JP-7206753 by Nikken Food KK, published Aug. 8, 1995; JP-5320025 by Kunimasa T. published Dec. 3, 1993; and JP-59157009 by Yakurigaku Chuou K E, published Sep. 6, 1984; among others; which patent publications are incorporated herein by reference.

Any of the compounds used according to a skin-darkening method of the present invention my be used in combination with “sunless tanning” agents as currently known in the art or to be developed in the future, including any one or more of those agents described in the following patent publications: U.S. Pat. No. 5,591,423 to Fuller; U.S. Pat. No. 5,628,987, to Fuller; EP 993826 by L'Oreal; and WO 99/56740, by Galderma Es. and Dev., among others, which patent publications are hereby incorporated in their entirety by reference.

For purposes of the present application, the terms “treatment”, “therapeutic use”, and “medicinal use” shall refer to any and all uses of the composition of the invention which remedy a disease state or one or more symptoms, or otherwise prevent, hinder, retard, or reverse the progression of disease or one or more other undesirable symptoms in any way whatsoever. For pharmaceutical uses, it is preferred that the compound that stimulates or inhibits pigmentation be part of a pharmaceutical composition.

Pharmaceutical compositions comprising an effective amount of a compound that inhibits or stimulates pigmentation function in a pharmaceutically acceptable carrier can be administered to a patient, person, or animal having a disease, disorder, or condition which is of a type that produces, or overproduces, melanin. The amount of a compound that inhibits or stimulates pigmentation which will be effective in the treatment of a particular disorder, disease, or condition will depend on the nature of the disease, disorder or condition, and can be determined by standard clinical techniques. Where possible, it is desirable to determine in vitro the cytotoxicity of the compound to the tissue type to be treated, and then in a useful animal model system prior to testing and use in humans.

The compounds of the present invention can be administered for the reduction or increase in pigmentation by any means that results in contact of the active agent with its site of action in the body of a mammal. The compounds can be administered by any conventional means available for use in conjunction with pharmaceuticals, either as individual therapeutic agents or in a combination of therapeutic agents. Each can be administered alone, but is preferably administered with a pharmaceutical carrier selected on the bases of the chosen route of administration and standard pharmaceutical practice. The pharmaceutical composition of the invention can be adapted for oral, parenteral, topical, or rectal administration, and can be in unit dosage form, in a manner well known to those skilled in the pharmaceutical art. Parenteral administration includes, but is not limited to, injection subcutaneously, intravenously, intraperitoneally, or intramuscularly. However, topical application is preferred.

In addition to pharmaceutical uses, the methods of the current invention are useful for cosmetic purposes. Cosmetic applications for methods of the present invention include the topical application of compositions containing one or more compounds that affect or inhibit pigmentation to enhance or otherwise alter the visual appearance of skin or hair. Occurrences in the skin or hair of noticeable but undesired pigmentation can be treated using the methods of the present invention.

An effective dosage and treatment protocol can be determined by conventional means, starting with a low dose in laboratory animals, and then increasing the dosage while monitoring the effects, and systematically varying the dosage regimen as well. Animal studies, preferably mammalian studies, are commonly used to determine the maximal tolerable dose, or MTD, of a bioactive agent per kilogram weight. Those skilled in the art can extrapolate doses for efficacy and avoidance of toxicity to other species, including humans.

Before human studies of efficacy are undertaken, Phase I clinical studies in normal subjects can help to establish safe doses. Numerous factors can be taken into consideration by a clinic when determining an optimal dosage for a given subject. Primary among these is the toxicity and half-life of the chosen compound that inhibits or stimulates pigment production. Additional factors include the size of the patient, the age of the patient, the general condition of the patient, the particular disease, condition, or disorder being treated, the severity of the disease, and the like. The trial dosages would be chosen after consideration of the results of animal studies and the clinical literature.

One of ordinary skill in the art will appreciate that the endpoint chosen in a particular case will vary according to the disease, condition, or disorder being treated, the outcome desired by the patient, subject, or treating physician, and other factors. Where the composition is being used to lighten or darken skin color such as, for example, to reverse hyperpigmentation caused by, for example, inflammation or diseases such as melasma, or to lighten or darken hair color, any one or a number of endpoints can be chosen. For example, endpoints can be defined subjectively such as, for example when the subject is simply “satisfied” with the results of the treatment. For pharmacological compositions, the endpoint can be determined by the patients or by the treating physician's, satisfaction with the results of the treatment. Alternatively, endpoints can be defined objectively. For example, the patient's or subject's skin or hair in the treated area can be compared to a color chart. Treatment is terminated when the color of the skin or hair in the treated area is similar in appearance to a color on the chart. Alternatively, the reflectance of the treated skin or hair can be measured, and treatment can be terminated when the treated skin or hair attains a specified reflectance. In another method, the amount of melanin in the skin or hair can be measured.

Treatment can be terminated when the melanin content of the treated hair or skin reaches a specified value. Melanin content can be determined in any way known in the art, including by histological methods, with or without enhancement by stains for melanin.

The compounds of the present invention can be administered orally in solid or semi-solid dosage forms, such as hard or soft-gelatin capsules, tablets, or powders, or in liquid dosage forms, such as elixirs, syrups, or suspensions. The compounds can also be administered pareneterally, in sterile liquid dosage forms. Since topical application is preferred, other dosage forms are possible including mousse or foams, patches, ointments, creams, gels, lotions, solutions, suppositories, or formulation for transdermal administration. Because in vivo use is contemplated, the composition is preferably of high purity and substantially free of potentially harmful contaminants, e.g., at least National Food grade, generally at least analytical grade, and preferably at least pharmaceutical grade. To the extent that a given compound must be synthesized prior to use, such synthesis or subsequent purification shall preferably result in a product that is substantially free of any potentially contaminating toxic agent that may have been used during the synthesis or purification process.

Gelatin capsules or liquid-filled soft gelatin capsules can containing the active ingredient, and powdered or liquid carriers, such as lactose, lecithin, starch, cellulose derivatives, magnesium stearate, stearic acid, and the like can be used. Similar diluents can be used to make compressed tablets. Both tablets and capsules can be manufactured as sustained release products to provide for continuous release of medication over a period of hours. Compressed tablets can be sugar-coated or film-coated to mask any unpleasant taste and to protect the tablet from the atmosphere, or enteric coated for selective, targeted disintegration in the gastrointestinal tract. Liquid dosage forms for oral administration can contain coloring and/or flavoring to increase patient acceptance.

In general, sterile water, oil, saline, aqueous dextrose, polysorbate, and related sugar solutions and glycols such as propylene glycol or polyethylene glycols, are suitable carriers for parenteral solutions. Solutions or emulsions for parenteral administration preferably contain about 5-15% polysorbate 80 or lecithin, suitable stabilizing agents, and, if necessary, substances. Antioxidizing agents, such as but not limited to sodium bisulfite, sodium sulfite, or ascorbic acid, either alone or combined, are suitable stabilizing agents. Also useful are citric acid and its salts, and sodium EDTA. In addition, parenteral solutions can contain preservatives including but not limited to benzalkonium chloride, methyl-or propyl paraben, and chlorobutanol. Suitable pharmaceutical carriers are further described in Remington's Pharmaceutical Sciences, 17th Edition, Merck Publishing Company, Easton, Pa. (1990), a standard reference text in this field, which is incorporated herein by reference in its entirety.

For topical administration, the compounds of the present invention can be formulated as a foam or mousse, solution, gel, lotion, ointment, cream, suspension, paste, liniment, powder, tincture, aerosol, transdermal drug delivery system, or the like, in a pharmaceutically or cosmetically acceptable form by methods well known in the art. The composition can be in any of a variety of forms common in the pharmaceutical or domestic arts for topical application to animals or humans, including solutions, lotions, sprays, creams, ointments, salves, gels, etc. Preferred agents are those that are viscous enough to remain on the treated area, those that do not readily evaporate, and/or those that are easily removed by rinsing with water topically with the aid of soaps, cleansers, and/or shampoos. Actual methods for preparing topical formulations are known or apparent to those skilled in the art, and are described in detail in Remington's Pharmaceutical Sciences (above) and Pharmaceutical Dosage Forms and Drug Delivery Systems, 6th Edition, Williams & Wilkins (1995).

Topical compositions according to the present invention may also include optional components, which should be suitable for application to keratinous tissue, that is, when incorporated into the composition, they are suitable for use in contact with human keratinous tissue without undue toxicity, incompatibility, instability, allergic response, and the like within the scope of sound medical judgment. In addition, such optional components are useful provided that they do not unacceptably alter the benefits of the active compounds of the invention. The CTFA Cosmetic Ingredient Handbook, Second Edition (1992) describes a wide variety of non-limiting cosmetic and pharmaceutical ingredients commonly used in the skin care industry, which are suitable for use in the compositions of the present invention. Examples of these ingredient classes include abrasives, absorbents, aesthetic components such as fragrances, pigments, colorings, essential oils, skin sensates, astringents etc, (e.g., clove oil, menthol, camphor, eucalyptus oil, eugenol, menthyl lactate, with hazel distillate, anti-acne agents, anti-caking agents, antifoaming agents, antimicrobial agents, antioxidants, binders, biological additives, buffering agents, bulking agents, chelating agents, chemical additives, colorants, cosmetic astringents cosmetic biocides, denaturants, drug astringents, external analgesics, film formers or materials such as polymers for aiding the film-forming properties and substantitivity of the composition (e.g., copolymer of eicosene and vinyl pyrrolidone), opacifying agents, pH adjusters, propellants reducing agents, sequestrants and/or healing agents (e.g., panthenol and derivatives such as ethyl panthenol), aloe vera, pantothenic acid and its derivatives, allantoin, and bisabolol), skin treating agents, thickeners, and vitamins and derivatives thereof.

In addition to the pharmaceutically effective amount of active compound disclosed therein, the topical compositions of the present invention also comprise a dermatologically acceptable carrier. The phrase “dermatologically acceptable carrier”, as used herein, means that the carrier is suitable for topical application to the skin, i.e., keratinous tissue has good aesthetic properties, is compatible with the active agents of the present invention and other components, and will not cause any safety or toxicity concerns. A safe and effective amount of carrier is from about 59% to about 99.99%, preferably from about 80% to about 99.99%, more preferably from about 90% to about 98%, and most preferably from about 90% to about 95% of the composition. The carrier used in the compositions of the present invasion can be in a wide variety of forms, including emulsion carriers, including, but not limited to, oil-in-water, water-in-oil, and oil-in-water-in-silicone emulsions, creams ointments, ophthalmic ointments, aqueous solution, lotions, or aerosols. As will be understood by the skilled artisan, a given component will distribute primarily into either the water or oil/silicone phase, depending upon the water solubility/dispersibility of the component in question.

Emulsions according to the present invention generally contain a pharmaceutically effective amount of an agent disclosed herein and a lipid or oil. Lipids and oils may be derived from animals, plants, or petroleum, and can be natural or synthetic. Preferred emulsions also contain a humectant such as glycerin. Emulsions will preferably further contain from about 1% to about 10%, more preferably from about 2% to about 5%, of an emulsifier, based on the weight of the carriers. Emulsifiers may be ionic, anionic, or cationic. The emulsion may also contain an anti-foaming agent to minimize foaming upon application to the keratinous tissue. Anti-foaming agents include high molecular weight silicones and other materials well known in the art for such use.

Suitable emulsions may have a wide range of viscosities, depending upon the product form. Exemplary low viscosity emulsions, which are preferred, have a viscosity of about 50 centistokes or less, more preferably about 10 centistokes or less, most preferably about 5 centistokes or less. The emulsion may also contain anti-foaming agents to minimize foaming upon application to the skin.

Other preferred topical carriers include oil-in-water emulsions having a continuous aqueous phase and a hydrophobic, water-insoluble phase dispersed therein. Preferred oil-in-water emulsions comprise from about 25% to about 98%, preferably from about 65% to about 95%, and more preferably from about 70% to about 90% water by weight of the topical carrier.

The hydrophobic phase is dispersed in the continuous aqueous phase. The hydrophobic phase may contain water insoluble or partially soluble materials such as are known in the art, including but not limited to silicones. The topical compositions of the present invention include, but are not limited to lotions and creams, and may comprise a dermatologically acceptable emollient. As used herein, “emollient” refers to a material useful for preventing or relieving dryness, as well as for protecting the skin. A wide variety of suitable emollients are known and may be used herein, including, for example, Sagarin, Cosmetics, Science, and Technology 2nd Edition Vol. 1, pp 3243 (1972), which contains numerous examples of materials suitable for use as an emollient. A preferred emollient is glycerin. Glycerin is preferably used in an amount of from about 0.001 to about 20%, more preferably from about 0.01 to about 10%, and most preferably from about 0.1 to about 5%.

Lotions and creams according to the present invention generally comprise a solution carrier system and one or more emollients. Lotions typically comprise from about 1% to about 20%, preferably from about 5% to about 20%, of emollient; from about 50% to about 90%, preferably from about 60% to about 80%, water; and a pharmaceutically effective amount of an agent described herein.

Ointments of the present invention may comprise a simple carrier base of animal or vegetable oil or semi-solid water soluble carriers. Ointments may further comprise a thickening agent and/or an emollient. For example an emollient may comprise from about 2% to about 20% of an emollient, about 0.1 to about 2% of a thickening agent, and a pharmaceutically effective amount of a compounds according to the present invention. In order to enhance the percutaneous absorption of the active ingredients, one or more of a number of agents can be added to the topical formulations, including but not limited to dimethylsulfoxide (DMSO), dimethylacetamide, dimethylformamde, surfactants, azone, alcohol, acetone, propylene glycol and polyethylene glycol. In addition, physical methods can also be used to enhance transdermal penetration such as e.g., by iontophoresis or sonophoresis. Alternatively, or in addition, the composition may be delivered in liposomes.

The pharmaceutical compositions of the present invention can be applied directly to the skin. Alternatively, they can be delivered by various transdermal drug delivery systems, such as transdermal patches as known in the art.

The foregoing description of the specific embodiments of the present invention will so fully reveal the general nature of the invention that others can, by applying current knowledge, readily modify and/or adapt for various application such specific embodiments without undue experimentation and without departing from the generic concept. Therefore, such adaptations and modifications should and are intended to be comprehended within the meaning and range of equivalents of the disclosed embodiments. 11. It is to be understood that the phraseology or terminology employed herein is for the purpose of description and not of limitation. The means and materials for carrying out disclosed functions may take a variety of alternative forms without departing from the invention. Thus, the expressions “means to . . . ” and “means for . . . ” as may be found the specification above, and/or in the claims below, followed by a functional statement, are intended to define and cover whatever structural, physical, chemical, or electrical element or structures which may now or in the future exist for carrying out the recited function, whether or not precisely equivalent to the embodiment or embodiments disclosed in the specification above, and it is intended that such expressions be given their broadest interpretation.

### References

1. Orlow, S. J., 1998, in The Pigmentary System: Physiology and Pathophysiology 97, Oxford University Press, New York, Nordlund et al., eds.

2. Silvers, W. K., 1979, The Coat Colors of Mice: A Model for Mammalian Gene Action and Interaction, Springer-Berlag, Basel

3. Lerner, A. B. et al., 1950, Physiolo. Rev. 30:90-126

4. Korner and Pawelek, 1982, Science 217:1163-1165

5. Hearing, V. J., 1993, Am. J. Hum. Genet. 52:1-7

6. Durham-Pierre, D. et al., 1994 Nature Genetics 7: 176-179

7. Sviderskaya, E. V., et al., 1997, J. Invest. Dermatol. 190:30-34

8. Lamoreux, M. L., et al., 1995, Pigment Cell Res. 8: 263-70

9. Gardner, J. M., et al., 1992, Science 257: 112-124

10. Rinchik, E. M. et al., 1993, Nature 361: 72-76

11. Sviderskaya, E. V. et al., above

12. Rosemblat, S., et al., 1998, Exp. Cell Res. 239: 3440352

13. Gahl, W. A., et al., 1995, Pigment Cell Res. 8:229-233

14. Brilliant, M. H. et al., 1998, above

15. Chiu, E., et al., 1993, Exp. Eye Res. 57: 301-305

16. Russell, E. S., 1949, Genetics 34: 146-166

17. Orlow, S. J., et al., 1999, Exp. Eye Res. 68: 147-154

18. Tasaka, K. et al., 1998, Meth. Find. Expl. Clin. Pharmacol 20:99-109

19. Iida, K. et al., 1995, Planta Med. 61:425-428

20. Reish, O., et al., 1995, Am. J. Hum. Genet. 57:127-132

What is claimed is:

1. A method for screening for a test compound selected from the group consisting of trisubstituted triazines and agents that interact with prohibitin or mitochondrial ATPase to determine if the test compound is one that inhibits or stimulates pigmentation comprising:
a. determining the amount of melanin or tyrosinase in cells or extracts of cells;
b. treating the cells with a test compound; and
c. determining the amount of melanin or tyrosinase in the treated cells or extracts of cells;
d. wherein a change in the amount of melanin or tyrosinase in the cells or the extract of cells in the presence of the test compound as compared to the absence of the test compound indicates that the test compound is one that affects pigmentation.

2. The method according to claim 1 wherein the amount of melanin in the cells or the extract of cells in the presence of the test compound increases, indicating that the compound is a stimulator of pigmentation.

3. The method according to claim 1 wherein the amount of melanin in the cells or the extract of cells in the presence of the test compound decreases, indicating that the compound is an inhibitor of pigmentation.

4. A method for decreasing pigmentation in a subject, comprising administering to the subject an effective amount of at least one trisubstituted triazine compound that inhibits pigmentation.

5. The method according to claim 4 comprising further administering to the subject a protein kinase A inhibitor.

6. A method for increasing pigmentation in a subject comprising administering to the subject an effective amount of at least one compound selected from the group consisting of trisubstituted triazine compounds that stimulate pigmentation, oligomycin and derivatives thereof that stimulate pigmentation, and aurovertin and derivatives thereof that stimulate pigmentation, and compounds that interact with prohibitin.

7. The method according to claim 6 comprising further administering to the subject at least one member selected from the group consisting of cAMP elevating agents, MAP kinase inhibitors, and protein kinase C inhibitors.

8. A method for increasing pigmentation in a subject comprising administering to the subject an effective amount of at least one compound that interacts with mitochondrial ATPase.

9. The method according to claim 8 wherein the at least one compound that interacts with mitochondrial ATPase is selected from the group consisting of PPA, ADA, oligomycin, and aurovertin, and derivatives thereof that stimulate pigmentation.

10. A composition for increasing pigmentation comprising an effective amount of at least one compound that interacts with mitochondrial ATPase in a pharmaceutically or cosmetically acceptable carrier.

11. The composition according to claim 11 wherein the at least one compound that interacts with mitochondrial ATPase is selected from the group consisting of PPA, ADA, oligomycin, and aurovertin, and derivatives thereof that stimulate pigmentation.

12. A composition for increasing pigmentation comprising an effective amount of at least one compound selected from the group consisting of trisubstituted triazine compounds that stimulate pigmentation, oligomycin, and aurovertin and derivatives thereof that stimulate pigmentation, in a pharmaceutically or cosmetically acceptable carrier.

13. The composition according to claim 12 further including at least one member selected from the group consisting of cAMP elevating agents, MAP kinase inhibitors, and protein kinase C inhibitors.

14. The composition according to claim 12 further including a “self-tanning” compound.

15. The composition according to claim 12 wherein the trisubstituted triazine compound is selected from the group consisting of

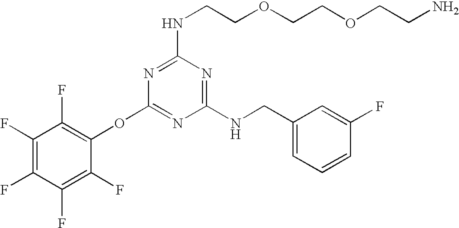

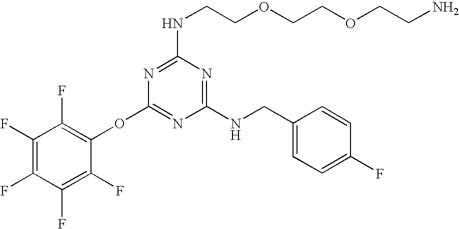

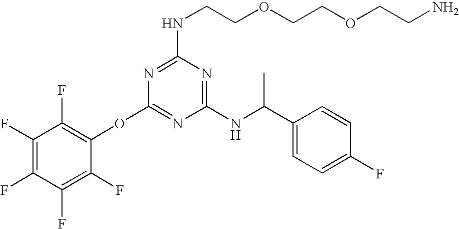

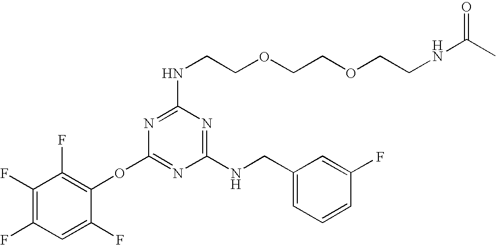

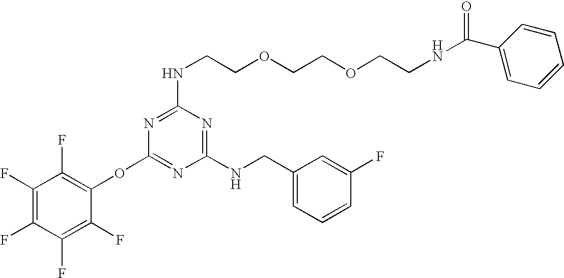

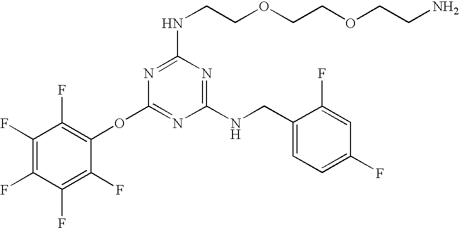

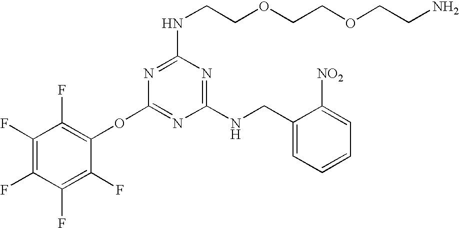

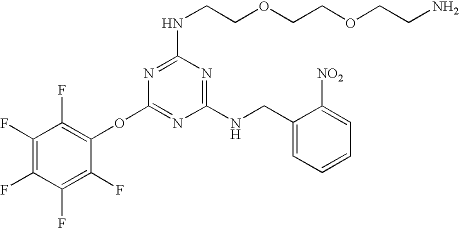

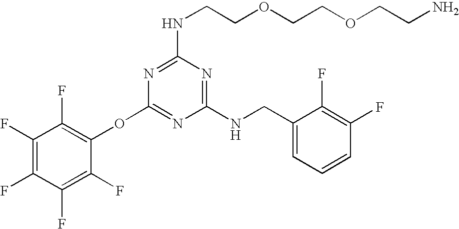

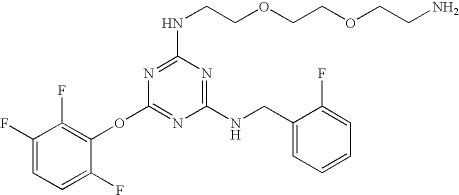

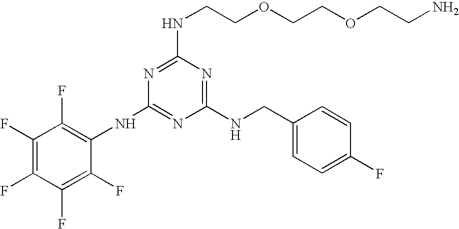

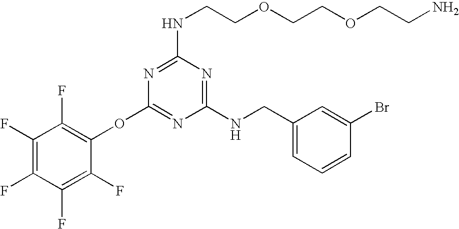

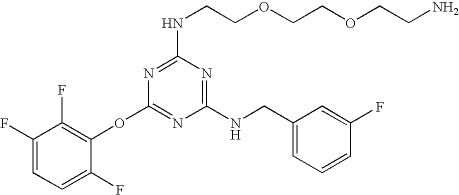

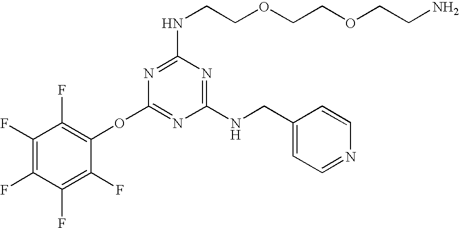

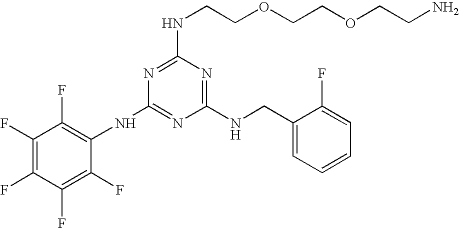

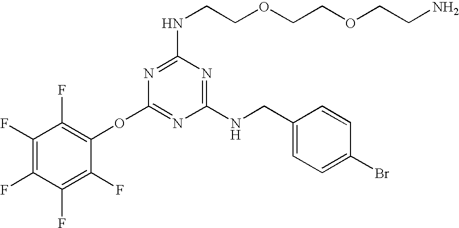

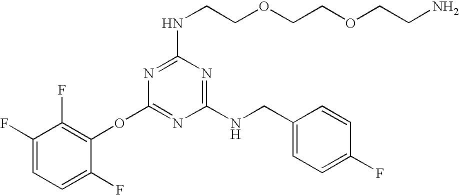

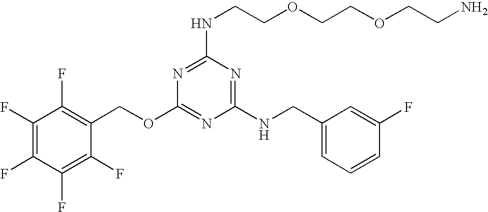

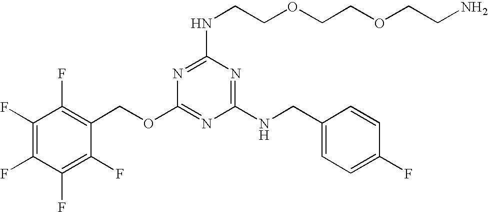

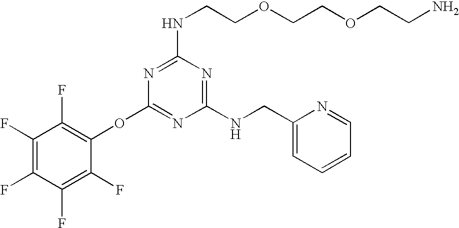

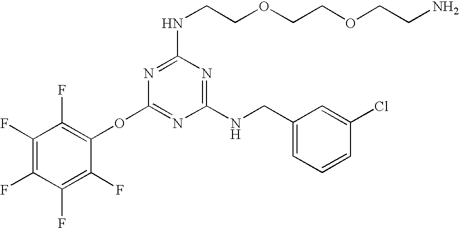

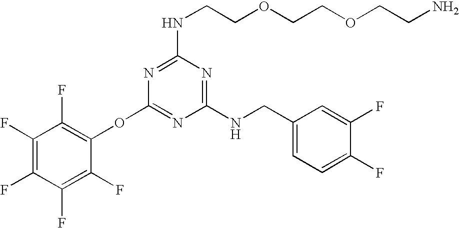

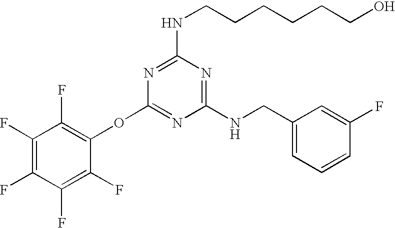

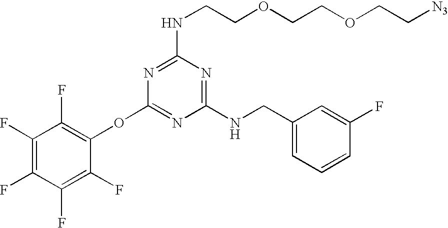

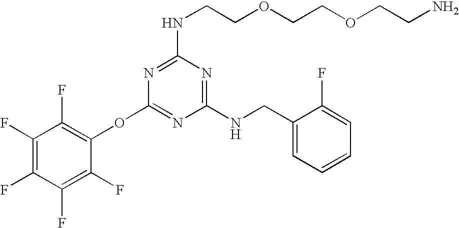

16. A method for screening for trisubstituted triazine compounds that affect pigmentation comprising:
a. identifying a trisubstituted triazine compound that affects pigmentation;
b. contacting a protein with the trisubstituted triazine compound and determining if the protein interacts with the trisubstituted triazine;
c. if the protein interacts with the trisubstituted triazine compound, using the protein as a target for other compounds that interact with the protein to determine if the other compounds also affect pigmentation.

17. The method according to claim 16 wherein the protein is prohibitin or a compound that binds with prohibitin.

18. A method for screening for trisubstituted triazines that affect prohibitin or molecules that bind to prohibitin comprising:
contacting prohibitin or a molecule that binds to prohibitin and determining if the prohibitin or molecule that binds to prohibitin interacts with the trisubstituted triazine.

19. A method for screening compounds to determine if the compounds affect pigmentation comprising:
a. incubating a compound to be tested with mitochondrial ATPase;
b. determining if the compound binds to ATPase; and
c. if the compound binds to ATPase, the compound is one which affects pigmentation.

20. The method according to claim 19 wherein the mitochondrial ATPase is in a form selected from an alpha, beta, or gamma subunit.

21. The method according to claim 19 wherein the compound to be tested is immobilized on a substrate.

22. The method according to claim 19 wherein the compound to be tested is tagged with a marker selected from the group consisting of fluorescent dyes, enzymes, and radiolabels.

23. The method according to claim 19 wherein binding to mitochondrial ATPase is determined by surface plasmon resonance.

24. A method for synthesizing a library of trisubstituted triazines comprising preparing three building blocks separately, and then assembling the building blocks by orthogonal reaction.

25. The method according to claim 24 wherein the building blocks comprise:
a. a primary amine of the formula RNH₂, wherein R is selected from the group consisting of substituted or unsubstituted alkyl, substituted or unsubstituted alkenyl, substituted or unsubstituted alkynyl, substituted or unsubstituted aryl, substituted or unsubstituted arylalkyl, and substituted or unsubstituted heteroaryl groups;
b. R₂NH, R₂OH, or R₂SH, wherein R₂ is selected from the group consisting of substituted or unsubstituted alkyl, substituted or unsubstituted alkenyl, substituted or unsubstituted alkynyl, substituted or unsubstituted aryl, substituted or unsubstituted arylalkyl, and substituted or unsubstituted heteroaryl groups; and
c. R₃R₃NH, R₃SH, wherein R₃ is selected from the group consisting of substituted or unsubstituted alkyl, substituted or unsubstituted alkenyl, substituted or unsubstituted alkynyl, substituted or unsubstituted aryl, substituted or unsubstituted arylalkyl, and substituted or unsubstituted heteroaryl groups.
